(19) United States
(12) Patent Application Publication
(10) Pub. No.: US 2022/0152019 A1
(43) Pub. Date: May 19, 2022

(54) COMPOUNDS, COMPOSITIONS, AND METHODS FOR TREATING INFLAMMATORY OR IMMUNE-MEDIATED CONDITIONS OF SURFACE TISSUES

(71) Applicant: Landos Biopharma, Inc., Blacksburg, VA (US)

(72) Inventors: Josep Bassaganya-Riera, Blacksburg, VA (US); Andrew Leber, Blacksburg, VA (US); Raquel Hontecillas, Blacksburg, VA (US)

(73) Assignee: Landos Biopharma, Inc., Blacksburg, VA (US)

(21) Appl. No.: 17/528,645

(22) Filed: Nov. 17, 2021

Related U.S. Application Data
(60) Provisional application No. 63/115,814, filed on Nov. 19, 2020.

(51) Int. Cl. A61K 31/496; A61P 17/06; A61P 17/00; A61K 9/00

(52) U.S. Cl. CPC A61K 31/496; A61P 17/06; A61K 9/0014; A61K 9/006; A61P 17/00

(57) ABSTRACT

Compounds, compositions, and methods for treating inflammatory or immune-mediated conditions of surface tissues. The methods preferably involve topically administering to a surface tissue an amount of a compound effective to treat an inflammatory or immune-mediated condition. The compounds include lanthionine synthetase C-like 2 (LANCL2) agonists, such as piperazine-1,4-diylbis((6-(1H-benzo[d]imidazo-2-yl)pyridine-2-yl)methanone) and salts and analogs thereof. The surface tissues include the skin and the mucosa, such as the oral mucosa. The inflammatory or immune-mediated conditions include psoriasis and dermatitis (such as atopic dermatitis), among others.

### FIELD OF THE INVENTION

The invention is directed to compounds, compositions, and methods for treating inflammatory or immune-mediated conditions of surface tissues. The invention is particularly directed lanthionine synthetase C-like 2 (LANCL2) agonists, compositions comprising same, and methods employing same for the treatment of inflammatory or immune-mediated conditions of surface tissues.

### BACKGROUND

Inflammatory and immune-mediated conditions of surface tissues such as the skin and mucosa are common. Many of the most common forms of immune-mediated inflammation of surface tissues are caused by pathogenic T cells, and many systemic T cell-mediated autoimmune diseases have associated surface-tissue manifestations. In particular, T cells have been implicated in the pathogenesis of psoriasis; atopic dermatitis; skin and mucosal lesions in Bechet's disease, scleroderma, systemic sclerosis, systemic lupus erythematosis, inflammatory bowel disease, Graves' disease, Hashimoto's thyroiditis, and graft-versus-host disease; drug induced hypersensitivity reactions; allergic contact dermatitis; vitiligo; dermatomyositis; bullous pemphigoid; pemphigus vulgaris; pemphigus foliaceus; lichen planus; fixed drug eruption; delayed-type hypersensitivity reaction; Vogt-Koyanagi-Harada syndrome; Sjogren's syndrome; alopecia areata; Wiskott-Aldrich syndrome; Stevens-Johnson syndrome; toxic epidermal necrolysis; cutaneous lupus erythematosis; lichenoid tissue reaction; and oral lichen planus. In addition, certain cutaneous T cell lymphomas are associated with aberrant memory T cells including mycosis fungoides and Sezary syndrome.

Steroids are widely prescribed to remediate the common inflammatory conditions of surface tissues. However, topical steroids can lose effectiveness with chronic use and can lead to specific dermatological side effects, including skin atrophy, rosacea, steroid allergy, skin irritation, and potentially mild Cushing's syndrome from systemic absorption. Second-line therapies have been developed for many diseases, and include, for example, topical tacrolimus and pimecrolimus, phototherapy, vitamin D analogs, retinoids, and the like. These topical therapies, however, often fail.

There is a significant unmet need for new topical therapies for the treatment of inflammatory or immune-mediated conditions of surface tissues.

### SUMMARY OF THE INVENTION

An aspect of the invention is directed to methods of treating inflammatory or immune-mediated conditions of surface tissues. The methods preferably comprise topically administering to a surface tissue an amount of a compound effective to treat the condition.

Another aspect of the invention is directed to compounds for use in the treatment of inflammatory or immune-mediated conditions of surface tissues. The treatments preferably comprise topically administering to a surface tissue an amount of a compound effective to treat the condition.

Another aspect of the invention is directed to uses of compounds for treating inflammatory or immune-mediated conditions of surface tissues. The treating preferably comprises topically administering to a surface tissue an amount of a compound effective to treat the condition.

Another aspect of the invention is directed to pharmaceutical compositions comprising a compound for treating inflammatory or immune-mediated conditions of surface tissues. The treating preferably comprises topically administering to a surface tissue an amount of a compound effective to treat the condition.

Another aspect of the invention is directed to uses of compounds for the manufacture of pharmaceutical compositions for the treatment of inflammatory or immune-mediated conditions of surface tissues. The treatment preferably comprises topically administering to a surface tissue an amount of a compound effective to treat the condition.

Another aspect of the invention is directed to uses of compounds for the preparation of medicaments for treatment of inflammatory or immune-mediated conditions of surface tissues. The treatment preferably comprises topically administering to a surface tissue an amount of a compound effective to treat the condition.

The compounds in the aspects of the invention comprise LANCL2 agonists. The LANCL2 agonists are preferably compounds of formula (I):

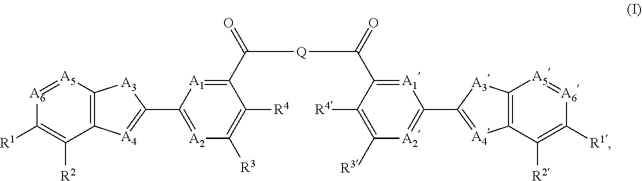

or a pharmaceutically acceptable salt thereof, wherein:
Q is piperazine-1,4-diyl; 2,5-diazabicyclo[2.2.1]heptane-2,5-diyl; 2,5-diazabicyclo[2.2.2]octane-2,5-diyl; 1,4-diazepane-1,4-diyl; benzene-1,4-diamine-N¹,N⁴-diyl; ethane-1,2-diamine-N¹,N²-diyl; N¹,N²-dialkylethane-1,2-diamine-N¹,N²-diyl; propane-1,3-diamine-N¹,N³-diyl; N¹,N³-dialkylpropane-1,3-diamine-N¹,N³-diyl; 1,4-diaminoanthracene-9,10-dione-1,4-diyl; C₆ arene-1,4-diamine-N¹,N⁴-diyl wherein the arene is substituted with one to four substituents independently selected from the group consisting of halogen, hydroxyl, carboxyl, optionally substituted alkyl, optionally substituted alkoxy, optionally substituted alkyloxycarbonyl, and optionally substituted aryl; or substituted piperazine-1,4-diyl wherein the piperazine is substituted with one to eight substituents independently selected from the group consisting of halogen, hydroxyl, carboxyl, optionally substituted alkyl, optionally substituted alkoxy, optionally substituted alkyloxycarbonyl, and optionally substituted aryl;
A₁ and A₁′ are each independently N or CR⁶;
A₂ and A₂′ are each independently N or CR⁷;
A₃ is NR⁸;
A₃′ is NR⁸, O, or S;
A₄ and A₄′ are each independently N or CR⁹;
A₅ and A₅′ are each independently N or CR¹⁰;
A₆ and A₆′ are each independently N or CR¹¹; and
R¹, R^{1′}, R², R^{2′}, R³, R^{3′}, R⁴, R^{4′}, R⁶, R⁷, R⁸, R⁹, R¹⁰, and R¹¹, if present, are in each instance independently hydrogen, halogen, optionally substituted alkyl, optionally substituted alkenyl, optionally substituted alkynyl, optionally substituted cycloalkyl, optionally substituted cycloalkenyl, hydroxyl, carboxyl, optionally substituted alkyloxy, optionally substituted alkenyloxy, optionally substituted alkynyloxy, optionally substituted cycloalkyloxy, optionally substituted cycloalkenyloxy, thiol, optionally substituted alkylthio, optionally substituted alkenylthio, optionally substituted alkynylthio, optionally substituted alkylsulfinyl, optionally substituted alkylsulfonyl, optionally substituted alkylsulfonyloxy, optionally substituted cycloalkylthio, optionally substituted cycloalkylsulfinyl, optionally substituted cycloalkylsulfonyl, optionally substituted cycloalkylsulfonyloxy, optionally substituted cycloalkenylthio, optionally substituted cycloalkenylsulfinyl, optionally substituted cycloalkenylsulfonyl, optionally substituted cycloalkenylsulfonyloxy, optionally substituted amino, acyl, optionally substituted alkyloxycarbonyl, optionally substituted alkenyloxycarbonyl, optionally substituted alkynyloxycarbonyl, optionally substituted aryloxycarbonyl, optionally substituted carbamoyl, optionally substituted sulfamoyl, cyano, nitro, optionally substituted aryl, optionally substituted aryloxy, optionally substituted arylthio, optionally substituted arylsulfinyl, optionally substituted arylsulfonyl, optionally substituted arylsulfonyloxy, optionally substituted heteroaryl, optionally substituted heteroaryloxy, optionally substituted heteroarylthio, optionally substituted heteroarylsulfinyl, optionally substituted heteroarylsulfonyl, optionally substituted heteroarylsulfonyloxy, or an optionally substituted non-aromatic heterocyclic group.

Exemplary surface tissues in the aspects of the invention include the skin and the mucosa, such as the oral mucosa.

Exemplary inflammatory or immune-mediated conditions in the aspects of the invention include psoriasis, cutaneous lupus erythematosus, dermatomyositis, pemphigoid, pemphigus, scleroderma, vasculitis, epidermolysis bullosa acquisita, vitiligo, lichen planus, scleritis, dermatitis, erythema nodosum, pyoderma gangrenosum, skin fissures, acne, enterocutaneous fistula, skin tags, canker sores, acrodermatitis enteropathica, pyoderma vegetans, leukocytoclastic vasculitis, anal fissures, Sweet's syndrome, rosacea, alopecia, keratoderma blennorrhagica, rosacea, cold sores, urticaria, actinic keratosis, carbuncle, cellulitis, ichthyosis vulgaris, skin infection, malar rash, photosensitivity, livedo reticularis, livedo reticularis, oral and nasal ulcers, purpura, mucositis, hemorrhoids, burn, and sunburn.

The objects and advantages of the invention will appear more fully from the following detailed description of the preferred embodiment of the invention made in conjunction with the accompanying drawings.

### BRIEF DESCRIPTION OF THE DRAWINGS

FIGS. 1A-1E. Results of a mouse model of psoriasis topically treated with BT-11 or vehicle (DMSO) control, showing psoriasis area and severity index (PASI) scores (FIG. 1A), splenic proportions of TNF-positive CD4+ T cells (FIG. 1B), splenic proportions of IL-17-positive CD4+ T cells (FIG. 1C), splenic proportions of IL-21-positive CD4+ T cells (FIG. 1D), and splenic proportions of IL-6-positive CD45+ cells (FIG. 1E).

FIGS. 2A-2D. Results of a mouse model of atopic dermatitis topically treated with BT-11 or placebo cream, showing disease activity index scores (FIG. 2A), cervical lymph node proportions of IFNγ-positive CD4+ T cells (FIG. 2B), cervical lymph node proportions of IL-4-positive CD4+ T cells (FIG. 2C), and cervical lymph node proportions of IL-5-positive CD4+ T cells (FIG. 2D).

### DETAILED DESCRIPTION OF THE INVENTION

An aspect of the invention is directed to methods of treating an inflammatory or immune-mediated condition of a surface tissue. The methods preferably comprise topically administering to the surface tissue an amount of a compound effective to treat the condition.

“Surface tissue” as used herein refers to any tissue of the body exposed to the external environment or lining an internal body cavity except for the lungs, stomach, intestines, and rectum (such as the rectal mucosa). In some versions, the surface tissue comprises the skin. Exemplary skin surface tissues include skin of the scalp, forehead, face, cheeks, nose, lips, chin, outer ears, inner ears, neck, chest, axilla, arms, upper arms, elbows, forearms, hands, fingers, torso, waist, buttocks, anus, groin, labia majora, scrotum, glans, prepuce, penile shaft, legs, thighs, knees, calves, ankles, feet, and toes. In some versions, the surface tissue comprises mucosa, exemplary mucosa surface tissues include the mouth (oral mucosa), nostrils, eyelids, ureter, urethra, and urinary bladder. In some versions, the surface tissue comprises exposed parts of the eyes. Exemplary eye surface tissues comprise the sclera and the cornea.

“Topically administering,” “topical administration,” and the like refers to the application of a compound (whether encompassed within a carrier or freely exposed) directly on, to, or at a surface tissue to deliver the compound primarily to the surface tissue and elicit a local effect at the surface tissue. Topical administration is distinct from forms of administration, such as oral, intravenous, intramuscular, and intraperitoneal administration, in which the compound is delivered within the body primarily to sites other than the site of administration to have primary effects at such other sites. Topical administration is also distinct from administration via injection, such as subcutaneous, intradermal, intrathecal, epidural, or intraosseous injection, in which the compound is delivered directly to internal tissues by bypassing surface tissues. “Oral administration” as used herein refers to the indirect delivery of a compound to non-oral parts of the body via the mouth and does not refer to the direct application of a compound (whether encompassed within a carrier or freely exposed) to the inner surfaces of the mouth to elicit a local effect at the inner surfaces of the mouth.

“Treat,” “treating,” and the like, such as in the phrase “effective to treat the condition,” refers to the full or partial amelioration or prophylaxis of the condition. These terms may encompass reducing symptoms, ameliorating symptoms, alleviating symptoms, reducing the severity of symptoms, reducing the incidence of the condition, diminishing the extent of the condition, stabilizing (i.e., not worsening) the state of the condition, delaying onset of the condition, slowing progression of the condition, inducing remission (whether partial or total) of the condition, or any other change in the condition that improves the therapeutic outcome in the subject. Treating can include eliciting a clinically significant response without excessive levels of side effects.

Subjects treated in the methods of the invention include animals, such as humans and non-human vertebrates, including wild, domestic, and farm animals or mammals.

“Condition” refers to a disorder, disease, or any symptom of a disorder or disease.

“Inflammatory or immune-mediated condition” refers to a condition involving inflammation or resulting, at least in part, from the activity of the immune system. Exemplary inflammatory or immune-mediated conditions include psoriasis, cutaneous lupus erythematosus, dermatomyositis, pemphigoid, pemphigus, scleroderma, vasculitis, epidermolysis bullosa acquisita, vitiligo, lichen planus, scleritis, dermatitis, erythema nodosum, pyoderma gangrenosum, skin fissures, acne, enterocutaneous fistula, skin tags, canker sores, acrodermatitis enteropathica, pyoderma vegetans, leukocytoclastic vasculitis, anal fissures, Sweet's syndrome, rosacea, alopecia, keratoderma blennorrhagica, rosacea, cold sores, urticaria, actinic keratosis, carbuncle, cellulitis, ichthyosis vulgaris, skin infection, malar rash, photosensitivity, livedo reticularis, livedo reticularis, oral and nasal ulcers, purpura, mucositis, hemorrhoids, burn, and sunburn.

The inflammatory or immune-mediated condition can comprise an inflammatory or immune-mediated condition of skin. Exemplary inflammatory or immune-mediated conditions of skin include psoriasis, cutaneous lupus erythematosus, dermatomyositis, pemphigoid, pemphigus, scleroderma, vasculitis, epidermolysis bullosa acquisita, vitiligo, lichen planus, scleritis, dermatitis, erythema nodosum, pyoderma gangrenosum, skin fissures, acne, enterocutaneous fistula, skin tags, acrodermatitis enteropathica, pyoderma vegetans, leukocytoclastic vasculitis, anal fissures, Sweet's syndrome, rosacea, alopecia, keratoderma blennorrhagica, rosacea, cold sores, urticaria, actinic keratosis, carbuncle, cellulitis, ichthyosis vulgaris, skin infection, malar rash, photosensitivity, livedo reticularis, annular erythema, purpura, hemorrhoids, burn, and sunburn.

The invention, the inflammatory or immune-mediated condition can comprise an inflammatory or immune-mediated condition of mucosa. Exemplary inflammatory or immune-mediated conditions of mucosa include cutaneous lupus erythematosus, dermatomyositis, pemphigoid, pemphigus, scleroderma, epidermolysis bullosa acquisita, lichen planus, pyoderma gangrenosum, canker sores, acrodermatitis enteropathica, pyoderma vegetans, anal fissures, Sweet's syndrome, cold sores, oral and nasal ulcers, mucositis, hemorrhoids, and burn.

The inflammatory or immune-mediated condition can comprise an inflammatory or immune-mediated condition of the oral mucosa. Exemplary immune-mediated conditions of the oral mucosa include cutaneous lupus erythematosus, dermatomyositis, pemphigoid, pemphigus, scleroderma, epidermolysis bullosa acquisita, lichen planus, pyoderma gangrenosum, canker sores, acrodermatitis enteropathica, pyoderma vegetans, Sweet's syndrome, cold sores, oral ulcers, oral mucositis, and burn.

The inflammatory or immune-mediated condition can comprise an autoimmune condition. “Autoimmune condition” refers to an autoimmune disease, an autoimmune disorder, or an inflammatory or immune-mediated condition of a surface tissue resulting from or linked to an underlying autoimmune disease or disorder. Exemplary autoimmune conditions include psoriasis, cutaneous lupus erythematosus, dermatomyositis, pemphigoid, pemphigus, scleroderma, vasculitis, epidermolysis bullosa acquisita, vitiligo, lichen planus, scleritis, dermatitis, erythema nodosum, pyoderma gangrenosum, skin fissures, acne, enterocutaneous fistula, skin tags, canker sores, acrodermatitis enteropathica, pyoderma vegetans, leukocytoclastic vasculitis, anal fissures, Sweet's syndrome, rosacea, and alopecia areata.

The treated subject can comprise a subject suffering from an autoimmune disease, such as inflammatory bowel disease (e.g., ulcerative colitis and Crohn's disease), celiac disease, systemic lupus erythematosus, rheumatoid arthritis, type 1 diabetes, psoriatic arthritis, and/or Sjögren syndrome. The autoimmune condition in such a case can comprise an immune-mediated condition of a surface tissue resulting from or linked to the underlying autoimmune disease. Exemplary immune-mediated conditions of surface tissues resulting from or linked to inflammatory bowel disease include pyoderma gangrenosum, skin fissures, anal fissures, acne, psoriasis, enterocutaneous fistulas, skin tags, canker sores, acrodermatitis enteropathica, pyoderma vegetans, leukocytoclastic vasculitis, vitiligo, clubbing, epidermolysis bullosa acquista, and Sweet's syndrome. Exemplary immune-mediated conditions of surface tissues resulting from or linked to celiac disease include dermatitis herpetiformis, eczema, psoriasis, urticaria, and alopecia areata. Exemplary immune-mediated conditions of surface tissues resulting from or linked to systemic lupus erythematosus include cutaneous lupus erythematosus (e.g., discoid lupus erythematosus), malar rash, photosensitivity, livedo reticularis, alopecia, oral and nasal ulcers, urticaria, purpura, and cutaneous vasculitis. Exemplary immune-mediated conditions of surface tissues resulting from or linked to rheumatoid arthritis include rheumatoid vasculitis and livedo reticularis. Exemplary immune-mediated conditions of surface tissues resulting from or linked to type 1 diabetes include scleroderma. Exemplary immune-mediated conditions of surface tissues resulting from or linked to psoriatic arthritis include psoriasis. Exemplary immune-mediated conditions of surface tissues resulting from or linked to Sjögren syndrome include vasculitis, annular erythema, livedo reticularis, erythema nodosum, alopecia, and vitiligo.

Aspects of the exemplary inflammatory or immune-mediated conditions described herein, including characteristics, identification, and diagnosis, are well known in the art. Several of the exemplary inflammatory or immune-mediated conditions are discussed below.

Psoriasis typically presents as lesions of plaques that are erythematous, thickened, and scaly. Current theory of the pathogenesis of psoriasis is that in individuals who are genetically susceptible a triggering event in the epidermis such as injury or super antigen contact initiates a response of the innate immune system with arachidonic acid and eicosanoid generation, recruitment and activity of neutrophils. Subsequent transformation of the response to that of a TH 1 adaptive immunity with cytokine activation and activity of specific T lymphocytes effect the pathological changes in the epidermis and dermis, which result in the typical psoriasis lesions. Exemplary forms of psoriasis include pustular psoriasis, guttate psoriasis, erythrodermic psoriasis, inverse psoriasis, and plaque psoriasis (psoriasis vulgaris).

Dermatitis (also called eczema) is generic inflammation of the skin. Exemplary forms dermatitis include atopic dermatitis, contact dermatitis, photo-induced dermatitis, dermatitis herpetiformis, dyshidrotic eczema, neurodermatitis, nummular eczema, pustular dermatitis, seborrheic dermatitis, and stasis dermatitis.

Contact dermatitis is an inflammatory condition of the skin either of irritant exposure to the skin without specific adaptive immunologic pathogenesis or of allergic sensitization and subsequent exposure of the skin to the sensitizing allergen with specific adaptive immunologic pathogenesis. Both involve innate and acquired immune system response including arachidonic acid and cytokine components that initiate and propagate the disease through cell to cell messaging by eicosanoid and/or cytokine moieties produced by epidermal cells, macrophages, dendritic cells, neutrophils, eosinophils, and various T and B lymphocytes. Contact dermatitis may be either acute or chronic. The acute forms are pruritic with erythema, edema, and micro or macrovesiculation in the areas of skin contact by the initiating factor. The chronic forms are pruritic with milder erythema, scaling, lichenification, and possibly fissuring, particularly on the hands.

Atopic dermatitis is typically part of the broader disease complex of atopy that includes asthma, hay fever, and atopic dermatitis. Many individuals with atopic dermatitis have various mutations of the fillagrin gene that codes for an important epidermal structural protein that when defective, results in abnormal barrier function of the epidermis. The altered barrier allows exposure to multiple environmental allergens that are first recognized by innate immune responses involving arachidonic acid and eicosanoids and recruitment of eosinophils, mast cells, and other inflammatory cells that initiate an acute responses of itch, erythema, and subsequent scratching and additionally activate the adaptive immune responses that involve inflammation by lymphocytes predominantly of a TH 2 derivation and activity.

Acne, (or acne vulgaris) is an inflammatory disorder of the pilosebaceous follicular unit especially of the face and upper chest and back. It is a very common disease of both males and females after initiation of puberty, and in females even prior to adrenal gland maturity. Increased production of androgenic hormones by adrenal, ovarian, and testicular glands and by the pilosebaceous unit itself produce an increase in sebum and changes in its lipid composition, which combine with follicular epithelial cells to produce some degree of obstruction of the infra-infundibular portion of the pilosebaceous follicle resulting in the initial lesion of acne, the microcomedo. This consequent dilation of the pore and the changed composition of sebum at puberty facilitate colonization of the follicle by Propionibacterium acnes bacilli that produce enzymes to degrade the triglycerides in sebum to free fatty acids that leak through the follicle into the dermis and incite arachidonic acid pathways of eicosanoid production and subsequent initiation of inflammation. The bacilli also initiate chemokine production that attracts further inflammatory cells to the area and consequent cytokine production and action to continue and amplify inflammation. Thus, initiation and propagation of progressive inflammation in the microcomedo produces the evolution to the several hallmark lesions of inflammatory acne, papule, pustule, nodule, and cyst. Exemplary types of acne treatable with the methods of the invention comprise common acne, comedonic acne, papulopustular acne, papulocomedonic acne, nodulocystic acne, acne conglobata, cheloid acne of the nape of the neck, recurrent miliary acne, necrotic acne, neonatal acne, occupational acne, acne rosacea, senile acne, solar acne, and acne medicamentosa.

Rosacea is a chronic condition characterized by facial erythema and sometimes pimples. Rosacea typically begins as redness on the central face across the cheeks, nose, or forehead, but can also less commonly affect the neck, chest, ears, and scalp. In some cases, additional symptoms, such as semi-permanent redness, telangiectasia (dilation of superficial blood vessels on the face), red domed papules (small bumps) and pustules, red gritty eyes, burning and stinging sensations, and in some advanced cases, a red lobulated nose (rhinophyma), may develop. There are 3 subtypes of rosacea that affect the skin: erythematotelangiectatic rosacea, papulopustular rosacea, and phymatous rosacea.

Cutaneous lupus erythematosus encompasses a wide range of dermatologic manifestations. Cutaneous lupus is divided into several subtypes, including acute cutaneous lupus erythematosus, subacute cutaneous lupus erythematosus, and chronic cutaneous lupus erythematosus. Chronic cutaneous lupus erythematosus includes discoid lupus erythematosus, lupus erythematosus profundus, chilblain cutaneous lupus, and lupus tumidus.

Pemphigoid is a family of autoimmune conditions that causes blistering and rashes on the skin and mucous membranes. The body mistakenly sends antibodies to bind to cells in the skin. These antibodies trigger a chain reaction that separates the bottom layer of cells from above layers. Exemplary forms of pemphigoid include mucous membrane pemphigoid (cicatricial pemphigoid) and bullous pemphigoid.

Pemphigus is a rare skin disorder characterized by blistering of the skin and mucous membranes. Exemplary forms of pemphigus include pemphigus vulgaris, pemphigus foliaceous, pemphigus erythematosus, drug-induced pemphigus, IgA pemphigus, pemphigus vegetans, and paraneoplastic pemphigus.

Mucositis is the painful inflammation and ulceration of the mucous membranes, usually as an adverse effect of chemotherapy and/or radiotherapy. Particular forms of mucositis include oral mucositis, which is inflammation and ulceration that occurs in the mouth.

Some aspects of the invention are directed to methods of treating an inflammatory or immune-mediated condition of the rectal mucosa, the methods comprise topically administering to the rectal mucosa an amount of a compound effective to treat the condition. The inflammatory or immune-mediated condition of the rectal mucosa can comprise one or more of cutaneous lupus erythematosus, pemphigoid, pemphigus, scleroderma, epidermolysis bullosa acquisita, lichen planus, pyoderma gangrenosum, rectal fissures, mucositis, hemorrhoids, proctitis, and inflammation of the rectum.

The compounds used in the methods of the invention can comprise any compound described in U.S. Pat. No. 9,556,146 to Bassaganya-Riera et al.; U.S. Pat. No. 9,839,635 to Bassaganya-Riera et al.; U.S. Pat. No. 10,028,950 to Bassaganya-Riera et al.; U.S. Pat. No. 10,201,538 to Bassaganya-Riera et al.; U.S. Pat. No. 10,493,072 to Bassaganya-Riera et al.; U.S. Pat. No. 10,682,349 to Bassaganya-Riera et al.; U.S. Pat. No. 10,849,895 to Bassaganya-Riera et al.; and US 2019/0160100 A1 to Bassaganya-Riera et al., each of which is incorporated herein by reference in its entirety.

The compounds used in the methods of the invention can comprise a compound of formula (I):

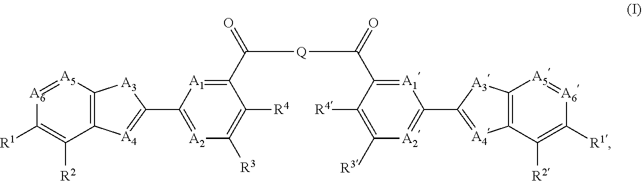

or a pharmaceutically acceptable salt thereof, wherein:
Q is piperazine-1,4-diyl; 2,5-diazabicyclo[2.2.1]heptane-2,5-diyl; 2,5-diazabicyclo[2.2.2]octane-2,5-diyl; 1,4-diazepane-1,4-diyl; benzene-1,4-diamine-N¹,N⁴-diyl; ethane-1,2-diamine-N¹,N²-diyl; N¹,N²-dialkylethane-1,2-diamine-N¹,N²-diyl; propane-1,3-diamine-N¹,N³-diyl; N¹,N³-dialkylpropane-1,3-diamine-N¹,N³-diyl; 1,4-diaminoanthracene-9,10-dione-1,4-diyl; C₆ arene-1,4-diamine-N¹,N⁴-diyl wherein the arene is substituted with one to four substituents (at the 2, 3, 5, and/or 6 positions) independently selected from the group consisting of halogen, hydroxyl, carboxyl, optionally substituted alkyl, optionally substituted alkoxy, optionally substituted alkyloxycarbonyl, and optionally substituted aryl; or substituted piperazine-1,4-diyl wherein the piperazine is substituted with one to eight substituents (at the 2, 3, 5, and/or 6 positions) independently selected from the group consisting of halogen, hydroxyl, carboxyl, optionally substituted alkyl, optionally substituted alkoxy, optionally substituted alkyloxycarbonyl, and optionally substituted aryl;
A₁ and A₁′ are each independently N or CR⁶;
A₂ and A₂′ are each independently N or CR⁷;
A₃ is NR⁸;
A₃′ is NR⁸, O, or S;
A₄ and A₄′ are each independently N or CR⁹;
A₅ and A₅′ are each independently N or CR¹¹;
A₆ and A₆′ are each independently N or CR¹¹; and
R¹, R^{1′}, R², R^{2′}, R³, R^{3′}, R⁴, R^{4′}, R⁶, R⁷, R⁸, R⁹, R¹⁰, and R¹¹, if present, are in each instance independently hydrogen, halogen, optionally substituted alkyl, optionally substituted alkenyl, optionally substituted alkynyl, optionally substituted cycloalkyl, optionally substituted cycloalkenyl, hydroxyl, carboxyl, optionally substituted alkyloxy, optionally substituted alkenyloxy, optionally substituted alkynyloxy, optionally substituted cycloalkyloxy, optionally substituted cycloalkenyloxy, thiol, optionally substituted alkylthio, optionally substituted alkenylthio, optionally substituted alkynylthio, optionally substituted alkylsulfinyl, optionally substituted alkylsulfonyl, optionally substituted alkylsulfonyloxy, optionally substituted cycloalkylthio, optionally substituted cycloalkylsulfinyl, optionally substituted cycloalkylsulfonyl, optionally substituted cycloalkylsulfonyloxy, optionally substituted cycloalkenylthio, optionally substituted cycloalkenylsulfinyl, optionally substituted cycloalkenylsulfonyl, optionally substituted cycloalkenylsulfonyloxy, optionally substituted amino, acyl, optionally substituted alkyloxycarbonyl, optionally substituted alkenyloxycarbonyl, optionally substituted alkynyloxycarbonyl, optionally substituted aryloxycarbonyl, optionally substituted carbamoyl, optionally substituted sulfamoyl, cyano, nitro, optionally substituted aryl, optionally substituted aryloxy, optionally substituted arylthio, optionally substituted arylsulfinyl, optionally substituted arylsulfonyl, optionally substituted arylsulfonyloxy, optionally substituted heteroaryl, optionally substituted heteroaryloxy, optionally substituted heteroarylthio, optionally substituted heteroarylsulfinyl, optionally substituted heteroarylsulfonyl, optionally substituted heteroarylsulfonyloxy, or an optionally substituted non-aromatic heterocyclic group.
   This definition of the compound of formula (I) explicitly includes any hydrate, solvate, micronized form, crystalline form, co-crystalline form, amorphous form, or polymorph of the compound or salt thereof. In some versions of the compound, Q is piperazine-1,4-diyl or substituted piperazine-1,4-diyl wherein the piperazine in the substituted piperazine-1,4-diyl is substituted with one to eight substituents independently selected from the group consisting of (C₁ to C₆)alkyl, aryl, aryl(C₁ to C₆)alkyl, C(O)OH, and C(O)O(C₁ to C₆)alkyl. In some versions of the compound, Q is piperazine-1,4-diyl. In some versions of the compound, A₁ is N. In some versions of the compound, A₁′ is N. In some versions of the compound, A₂ is CR⁷. In some versions of the compound, A₂′ is CR⁷. In some versions of the compound, A₃′ is NR⁸ or O. In some versions of the compound, A₃′ is NR⁸. In some versions of the compound, A₄ is N. In some versions of the compound, A₄′ is N. In some versions of the compound, A₅ is CR¹⁰. In some versions of the compound, A₅′ is CR¹⁰. In some versions of the compound, A₆ is CR¹¹. In some versions of the compound, A₆′ is CR¹¹. In some versions of the compound, R¹, R^{1′}, R², R^{2′}, R³, R^{3′}, R⁴, R^{4′}, R⁶, R⁷, R⁸, R⁹, R¹⁰, and R¹¹, if present, are in each instance independently hydrogen, halogen, optionally substituted C1-C6 alkyl, hydroxyl, carboxyl, optionally substituted cycloalkyl, optionally substituted C1-C6 alkyloxy, optionally substituted amino, acyl, optionally substituted alkyloxycarbonyl, optionally substituted aryl, optionally substituted heteroaryl, or optionally substituted non-aromatic heterocyclic group. In some versions of the compound, R¹, R^{1′}, R², R^{2′}, R³, R^{3′}, R⁴, R^{4′}, R⁶, R⁷, R⁸, R⁹, R¹⁰, and R¹¹, if present, are in each instance independently hydrogen, halogen, unsubstituted C1-C6 alkyl, hydroxyl, carboxyl, unsubstituted cycloalkyl, unsubstituted C1-C6 alkyloxy, unsubstituted amino, acyl, unsubstituted alkyloxycarbonyl, unsubstituted aryl, unsubstituted heteroaryl, or unsubstituted non-aromatic heterocyclic group. In some versions of the compound, R¹, R^{1′}, R², R^{2′}, R³, R^{3′}, R⁴, R^{4′}, R⁶, R⁷, R⁸, R⁹, R¹⁰, and R¹¹, if present, are in each instance independently hydrogen or halogen. In some versions of the compound, R¹, R^{1′}, R², R^{2′}, R³, R^{3′}, R⁴, R^{4′}, R⁶, R⁷, R⁸, R⁹, R¹⁰, and R¹¹, if present, are each hydrogen.

In some versions of the compound:

each optionally substituted alkyl, optionally substituted alkyloxy, optionally substituted alkylthio, optionally substituted alkylsulfinyl, optionally substituted alkylsulfonyl, optionally substituted alkylsulfonyloxy, and optionally substituted alkyloxycarbonyl, when substituted, is independently substituted with one to three substituent(s) selected from the group consisting of cycloalkyl, alkylene optionally containing one or two heteroatom(s), hydroxy, oxo, alkyloxy optionally substituted with a substituent group A at one to three position(s), mercapto, alkylthio, a halogen atom, nitro, cyano, carboxy, alkyloxycarbonyl, optionally substituted amino, optionally substituted carbamoyl, acyl, aryl optionally substituted with a substituent group B at one to three position(s), heteroaryl optionally substituted with a substituent group C at one to three position(s), an optionally substituted non-aromatic heterocyclic ring group optionally substituted with a substituent group C at one to three position(s), aryloxy optionally substituted with a substituent group B at one to three position(s), and alkylsulfonyl;

each optionally substituted alkenyl, optionally substituted alkynyl, optionally substituted alkenyloxy, optionally substituted alkynyloxy, optionally substituted alkenylthio, optionally substituted alkynylthio, optionally substituted alkenyloxycarbonyl, optionally substituted alkynyloxycarbonyl, optionally substituted cycloalkyl, optionally substituted cycloalkenyl, optionally substituted cycloalkyloxy, optionally substituted cycloalkenyloxy, optionally substituted cycloalkylthio, optionally substituted cycloalkenylthio, optionally substituted cycloalkylsulfinyl, optionally substituted cycloalkenylsulfinyl, optionally substituted cycloalkylsulfonyl, optionally substituted cycloalkenylsulfonyl, optionally substituted cycloalkylsulfonyloxy, optionally substituted cycloalkenylsulfonyloxy, and optionally substituted alkylene optionally containing one or two heteroatom(s), when substituted, is independently substituted with one or more substituent(s) selected from the group consisting of alkyl optionally substituted with a substituent group D at one to three position(s), cycloalkyl, hydroxy, oxo, alkyloxy optionally substituted with a substituent group A at one to three position(s), mercapto, alkylthio, a halogen atom, nitro, cyano, carboxy, alkyloxycarbonyl, optionally substituted amino, optionally substituted carbamoyl, acyl, acyloxy, aryl optionally substituted with a substituent group B at one to three position(s), heteroaryl optionally substituted with a substituent group C at one to three position(s), non-aromatic heterocyclic group optionally substituted with a substituent group C at one to three position(s), aryloxy optionally substituted with a substituent group C at one to three position(s), and alkylsulfonyl;

each optionally substituted aryl, optionally substituted aryloxy, optionally substituted aryloxycarbonyl, optionally substituted arylthio, optionally substituted arylsulfinyl, optionally substituted arylsulfonyl, optionally substituted arylsulfonyloxy, optionally substituted heteroaryl, optionally substituted heteroaryloxy, optionally substituted heteroarylthio, optionally substituted heteroarylsulfinyl, optionally substituted heteroarylsulfonyl, optionally substituted heteroarylsulfonyloxy, and optionally substituted non-aromatic heterocyclic group, when substituted, are each independently substituted with one or more substituent(s) selected from the group consisting of alkyl optionally substituted with a substituent group D at one to three position(s), oxo, cycloalkyl, alkenyl, alkynyl, hydroxy, alkyloxy optionally substituted with a substituent group A at one to three position(s), aryloxy optionally substituted with a substituent group B at one to three position(s), mercapto, alkylthio, a halogen atom, nitro, cyano, carboxy, alkyloxycarbonyl, acyl, alkylsulfonyl, optionally substituted amino, optionally substituted carbamoyl, aryl optionally substituted with a substituent group B at one to three position(s), heteroaryl optionally substituted with a substituent group C at one to three position(s), and non-aromatic heterocyclic group optionally substituted with a substituent group C at one to three position(s);

each optionally substituted amino, optionally substituted carbamoyl, and optionally substituted sulfamoyl, when substituted, is independently substituted with one or two substituent(s) selected from the group consisting of alkyl, alkenyl, alkynyl, cycloalkyl, cycloalkynyl, aryl, heteroaryl, acyl, alkyloxycarbonyl, alkenyloxycarbonyl, alkynyloxycarbonyl, alkyl sulfonyl, alkenylsulfonyl, alkynylsulfonyl, arylsulfonyl, and heteroarylsulfonyl;

each substituent group A is independently selected from the group consisting of a halogen atom and phenyl optionally substituted with one to three substituent(s) selected from substituent group B;

each substituent group B is independently selected from the group consisting of a halogen atom, alkyl, alkyloxy, cyano, and nitro;

each substituent group C is independently selected from the group consisting of a halogen atom and alkyl; and

each substituent group D is independently selected from the group consisting of a halogen atom and alkyloxy.

In some versions, the compound has the structure of:

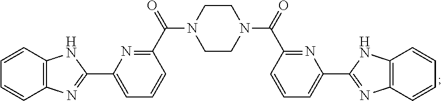 [SMILES: O=C(c1cccc(-c2nc3ccccc3[nH]2)n1)N1CCN(C(=O)c2cccc(-c3nc4ccccc4[nH]3)n2)CC1]

a pharmaceutically acceptable salt thereof. The structure shown above is a free base form of piperazine-1,4-diylbis((6-(1H-benzo[d]imidazo-2-yl)pyridine-2-yl)methanone). An exemplary dihydrochloride salt form of piperazine-1,4-diylbis((6-(1H-benzo[d]imidazo-2-yl)pyridine-2-yl)methanone) has the following structure:

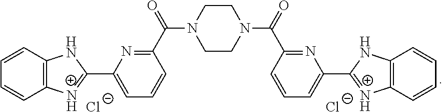 [SMILES: O=C(c1cccc(-c2[nH]c3ccccc3[nH+]2)n1)N1CCN(C(=O)c2cccc(-c3[nH]c4ccccc4[nH+]3)n2)CC1 ; [Cl-] ; [Cl-]]

Piperazine-1,4-diylbis((6-(1H-benzo[d]imidazo-2-yl)pyridine-2-yl)methanone), regardless of its form, is also referred to herein as “BT-11,” consistent with the current literature. See U.S. Pat. No. 9,556,146 to Bassaganya-Riera et al.; U.S. Pat. No. 9,839,635 to Bassaganya-Riera et al.; U.S. Pat. No. 10,028,950 to Bassaganya-Riera et al.; U.S. Pat. No. 10,201,538 to Bassaganya-Riera et al.; U.S. Pat. No. 10,493,072 to Bassaganya-Riera et al.; U.S. Pat. No. 10,682,349 to Bassaganya-Riera et al.; U.S. Pat. No. 10,849,895 to Bassaganya-Riera et al.; US 2019/0160100 A1 to Bassaganya-Riera et al.; Bissel et al. 2016 (Bissel P, Boes K, Hinckley J, Jortner B S, Magnin-Bissel G, Werre S R, Ehrich M, Carbo A, Philipson C, Hontecillas R, Philipson N, G andour RD, Bassaganya-Riera J. Exploratory Studies With BT-11: A Proposed Orally Active Therapeutic for Crohn's Disease. Int J Toxicol. 2016 September; 35(5):521-9); and Carbo et al. 2016 (Carbo A, Gandour R D, Hontecillas R, Philipson N, Uren A, Bassaganya-Riera J. An N,N-Bis(benzimidazolylpicolinoyl)piperazine (BT-11): A Novel Lanthionine Synthetase C-Like 2-Based Therapeutic for Inflammatory Bowel Disease. J Med Chem. 2016 Nov. 23; 59(22):10113-10126); Leber et al. 2018 (Leber A, Hontecillas R, Zoccoli-Rodriguez V, Bassaganya-Riera J. Activation of LANCL2 by BT-11 Ameliorates IBD by Supporting Regulatory T Cell Stability Through Immunometabolic Mechanisms. Inflamm Bowel Dis. 2018 Aug. 16; 24(9):1978-1991); Leber et al. 2019 Int J Toxicol. (Leber A, Hontecillas R, Zoccoli-Rodriguez V, Ehrich M, Davis J, Chauhan J, Bassaganya-Riera J. Nonclinical Toxicology and Toxicokinetic Profile of an Oral Lanthionine Synthetase C-Like 2 (LANCL2) Agonist, BT-11. Int J Toxicol 2019 March/April; 38(2):96-109); Leber et al. 2019 J Immunol (Leber A, Hontecillas R, Zoccoli-Rodriguez V, Chauhan J, Bassaganya-Riera J. Oral Treatment with BT-11 Ameliorates Inflammatory Bowel Disease by Enhancing Regulatory T Cell Responses in the Gut. J Immunol. 2019 Apr. 1; 202(7):2095-2104); and Leber et al. 2020 (Leber A, Hontecillas R, Zoccoli-Rodriguez V, Colombel J F, Chauhan J, Ehrich M, Farinola N, Bassaganya-Riera J. The Safety, Tolerability, and Pharmacokinetics Profile of BT-11, an Oral, Gut-Restricted Lanthionine Synthetase C-Like 2 Agonist Investigational New Drug for Inflammatory Bowel Disease: A Randomized, Double-Blind, Placebo-Controlled Phase I Clinical Trial, Inflamm Bowel Dis. 2020 Mar. 4; 26(4):643-652).

Exemplary crystalline forms of piperazine-1,4-diylbis((6-(1H-benzo[d]imidazo-2-yl)pyridine-2-yl)methanone) include Form 1, Form 2, Form 3, Form 4, Form 5, Form 6, Form 7, Form 8, Form 9, Form 10, Form 11, Form 12, and Form 13. See U.S. application Ser. No. 17/517,184.

In some versions, the compound has the structure of:

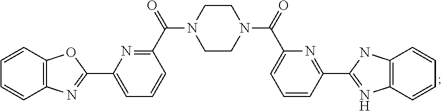 [SMILES: O=C(c1cccc(-c2nc3ccccc3[nH]2)n1)N1CCN(C(=O)c2cccc(-c3nc4ccccc4o3)n2)CC1]

a pharmaceutically acceptable salt thereof.

In some versions, the compound has the structure of:

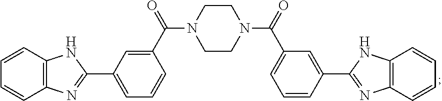 [SMILES: O=C(c1cccc(-c2nc3ccccc3[nH]2)c1)N1CCN(C(=O)c2cccc(-c3nc4ccccc4[nH]3)c2)CC1]

a pharmaceutically acceptable salt thereof.

“Pharmaceutically acceptable salts,” as used herein, are salts that retain the desired biological activity of the parent compound and do not impart undesired toxicological effects. Pharmaceutically acceptable salt forms include various crystalline polymorphs as well as the amorphous form of the different salts. The pharmaceutically acceptable salts can be formed with metal or organic counterions and include, but are not limited to, alkali metal salts such as sodium or potassium; alkaline earth metal salts such as magnesium or calcium; and ammonium or tetraalkyl ammonium salts, i.e., NX₄⁺ (wherein X is C1-4).

The term “halogen” refers to fluorine, chlorine, bromine, and iodine. Fluorine, chlorine, and bromine are preferred.

The term “hetero atom” refers to an oxygen atom, a sulfur atom, and a nitrogen atom.

The term “alkyl” includes a monovalent straight or branched hydrocarbon group having one to eight carbon atom(s). Examples include methyl, ethyl, n-propyl, isopropyl, n-butyl, isobutyl, sec-butyl, tert-butyl, n-pentyl, isopentyl, neo-pentyl, n-hexyl, isohexyl, n-heptyl, n-octyl, and the like. C1-C6 alkyl is preferred. C1-C4 alkyl or C1-C3 alkyl is further preferred. When a number of carbons is specified, it means “alkyl” having the carbon number within the range.

The term “alkenyl” includes a monovalent straight or branched hydrocarbon group having two to eight carbon atoms and one or more double bond(s). Examples include vinyl, allyl, 1-propenyl, 2-butenyl, 2-pentenyl, 2-hexenyl, 2-heptenyl, 2-octenyl, and the like. C2-C6 alkenyl is preferred. C2-C4 or C2-C3 alkenyl is further preferred.

The term “alkynyl” includes a monovalent straight or branched hydrocarbon group having two to eight carbon atoms and one or more triple bond(s). Examples include ethynyl, 1-propynyl, 2-propynyl, 2-butynyl, 2-pentynyl, 2-hexynyl, 2-heptynyl, 2-octynyl, and the like. C2-C6 alkynyl is preferred. C2-C4 or C2-C3 alkynyl is further preferred.

The term “cycloalkyl” includes a cycloalkyl having three to eight carbon atoms. Examples include cyclopropyl, cyclobutyl, cyclopentyl, cyclohexyl, cycloheptyl, cyclooctyl, and the like. C3-C6 cycloalkyl is preferred.

The term “cycloalkenyl” includes a cycloalkenyl having three to eight carbon atoms. Examples include cyclopropenyl, cyclobutenyl, cyclopentenyl, cyclohexenyl, cycloheptenyl, cycloocentyl, and the like. C3-C6 cycloalkenyl is preferred.

The term “alkyloxy” includes a group wherein an oxygen atom is substituted with one “alkyl” as described herein. Examples include methyloxy, ethyloxy, n-propyloxy, isopropyloxy, n-butyloxy, isobutyloxy, sec-butyloxy, tert-butyloxy, n-pentyloxy, isopentyloxy, 2-pentyloxy, 3-pentyloxy, n-hexyloxy, isohexyloxy, 2-hexyloxy, 3-hexyloxy, n-heptyloxy, n-octyloxy, and the like. C1-C6 alkyloxy is preferred. C1-C4 alkyloxy or C1-C3 alkyloxy is further preferred. When a number of carbons is specified, it means “alkyloxy” having the carbon number within the range.

The term “alkenyloxy” includes a group wherein an oxygen atom is substituted with one “alkenyl” as described herein. Examples include vinyloxy, allyloxy, 1-propenyloxy, 2-butenyloxy, 2-pentenyloxy, 2-hexenyloxy, 2-heptenyloxy, 2-octenyloxy, and the like. C2-C6 alkenyloxy is preferred. Moreover, C2-C4 or C2-C3 alkenyloxy is further preferred. When a number of carbons is specified, it means “alkenyloxy” having the carbon number within the range.

The term “alkynyloxy” includes a group wherein an oxygen atom is substituted with one “alkynyl” as described herein. Examples include ethynyloxy, 1-propynyloxy, 2-propynyloxy, 2-butynyloxy, 2-pentynyloxy, 2-hexynyloxy, 2-heptynyloxy, 2-octynyloxy, and the like. C2-C6 alkynyloxy is preferred. C2-C4 or C2-C3 alkynyloxy is further preferred. When a number of carbons is specified, it means “alkynyloxy” having the carbon number within the range.

The term “cycloalkyloxy” includes a group wherein an oxygen atom is substituted with one “cycloalkyl” as described herein. Examples include cyclopropyloxy, cyclobutyloxy, cyclopentyloxy, cyclohexyloxy, cycloheptyloxy, and cyclooctyloxy. C3-C6 cycloalkyloxy is preferred. When a number of carbons is specified, it means “cycloalkyloxy” having the carbon number within the range.

The term “cycloalkenyloxy” includes a group wherein an oxygen atom is substituted with one “cycloalkenyl” as described herein. Examples include cyclopropenyloxy, cyclobutenyloxy, cyclopentenyloxy, cyclohexenyloxy, cycloheptenyloxy, and cyclooctenyloxy. C3-C6 cycloalkenyloxy is preferred. When a number of carbons is specified, it means “cycloalkenyloxy” having the carbon number within the range.

The term “alkylthio” includes a group wherein a sulfur atom is substituted with one “alkyl” as described herein. Examples include methylthio, ethylthio, n-propylthio, isopropylthio, n-butylthio, isobutylthio, sec-butylthio, tert-butylthio, n-pentylthio, isopentylthio, 2-pentylthio, 3-pentylthio, n-hexylthio, isohexylthio, 2-hexylthio, 3-hexylthio, n-heptylthio, n-octylthio, and the like. C1-C6 Alkylthio is preferred. C1-C4 alkylthio is further preferred. C1-C3, C1-C2, or C1 alkylthio is further preferred. When a number of carbons is specified, it means “alkylthio” having the carbon number within the range.

The term “alkenylthio” includes a group wherein a sulfur atom is substituted with one “alkenyl” as described herein. Examples include vinylthio, allylthio, 1-propenylthio, 2-butenylthio, 2-pentenylthio, 2-hexenylthio, 2-heptenylthio, 2-octenylthio, and the like. C2-C6 Alkenylthio is preferred. C2-C4 or C2-C3 alkylthio is further preferred. When a number of carbons is specified, it means “alkenylthio” having the carbon number within the range.

The term “alkynylthio” includes a group wherein a sulfur atom is substituted with one “alkynyl” as described herein. Examples include ethynylthio, 1-propynylthio, 2-propynylthio, 2-butynylthio, 2-pentynylthio, 2-hexynylthio, 2-heptynylthio, 2-octynylthio, and the like. C2-C6 alkynylthio is preferred. C2-C4 or C2-C3 alkynylthio is further preferred. When a number of carbons is specified, it means “alkynylthio” having the carbon number within the range.

The term “alkylsulfinyl” includes a group wherein sulfinyl is substituted with one “alkyl” as described herein. Examples include methylsulfinyl, ethylsulfinyl, n-propylsulfinyl, isopropylsulfinyl, n-butylsulfinyl, isobutylsulfinyl, sec-butylsulfinyl, tert-butylsulfinyl, n-pentylsulfinyl, isopentylsulfinyl, 2-pentylsulfinyl, 3-pentylsulfinyl, n-hexylsulfinyl, isohexylsulfinyl, 2-hexylsulfinyl, 3-hexylsulfinyl, n-heptylsulfinyl, n-octylsulfinyl, and the like. C1-C6 alkylsulfinyl is preferred. C1-C4 or C1-C3 alkylsulfinyl is further preferred.

The term “alkylsulfonyl” includes a group wherein sulfonyl is substituted with one “alkyl” as described herein. Examples include methylsulfonyl, ethylsulfonyl, n-propylsulfonyl, isopropylsulfonyl, n-butylsulfonyl, isobutylsulfonyl, sec-butylsulfonyl, tert-butylsulfonyl, n-pentylsulfonyl, isopentylsulfonyl, 2-pentylsulfonyl, 3-pentylsulfonyl, n-hexylsulfonyl, isohexylsulfonyl, 2-hexylsulfonyl, 3-hexylsulfonyl, n-heptylsulfonyl, n-octylsulfonyl, and the like. C1-C6 alkylsulfonyl is preferred. C1-C4 or C1-C3 alkylsulfonyl is further preferred.

The term “alkylsulfonyloxy” includes a group wherein an oxygen atom is substituted with one “alkylsulfonyl” as described herein. Examples include methylsulfonyloxy, ethylsulfonyloxy, n-propylsulfonyloxy, isopropylsulfonyloxy, n-butylsulfonyloxy, isobutylsulfonyloxy, sec-butylsulfonyloxy, tert-butylsulfonyloxy, n-pentylsulfonyloxy, isopentylsulfonyloxy, 2-pentylsulfonyloxy, 3-pentylsulfonyloxy, n-hexylsulfonyloxy, isohexylsulfonyloxy, 2-hexylsulfonyloxy, 3-hexylsulfonyloxy, n-heptylsulfonyloxy, n-octylsulfonyloxy, and the like. C1-C6 alkylsulfonyl is preferred. C1-C4 or C1-C3 alkylsulfonyl is further preferred.

The term “cycloalkylthio” includes a group wherein a sulfur atom is substituted with one “cycloalkyl” as described herein. Examples include cyclopropylthio, cyclobutylthio, cyclopentylthio, cyclohexylthio, cycloheptylthio, cyclooctylthio, and the like. C3-C6 cycloalkylthio is preferred. When a number of carbons is specified, it means “cycloalkylthio” having the carbon number within the range.

The term “cycloalkylsulfinyl” includes a group in which sulfinyl is substituted with one “cycloalkyl” as described herein. Examples include cyclopropylsulfinyl, cyclobutylsulfinyl, cyclopentylsulfinyl, cyclohexylsulfinyl, cycloheptylsulfinyl, and cyclooctylsulfinyl. Preferably C3-C6 cycloalkylsulfinyl.

The term “cycloalkylsulfonyl” includes a group in which sulfonyl is substituted with one “cycloalkyl” as described herein. Examples include cyclopropylsulfonyl, cyclobutylsulfonyl, cyclopentylsulfonyl, cyclohexylsulfonyl, cycloheptylsulfonyl, and cyclooctylsulfonyl. C3-C6 cycloalkylsulfonyl is preferred.

The term “cycloalkylsulfonyloxy” includes a group in which an oxygen atom is substituted with one “cycloalkylsulfonyl” as described herein. Examples include cyclopropylsulfonyloxy, cyclobutylsulfonyloxy, cyclopentyl sulfonyloxy, cyclohexyl sulfonyloxy, cycloheptylsulfonyloxy, and cyclooctylsulfonyloxy. C6-C3 cycloalkylsulfonyloxy is preferred.

The term “cycloalkenylthio” includes a group in which a sulfur atom is substituted with one “cycloalkenyl” as described herein. Examples include cyclopropenylthio, cyclobutenylthio, cyclopentenylthio, cyclohexenylthio, cycloheptenylthio, and cyclooctenylthio. C3-C6 cycloalkenylthio is preferred. When a number of carbons is specified, it means “cycloalkenylthio” having the carbon number within the range.

The term “cycloalkenylsulfinyl” includes a group in which sulfinyl is substituted with one “cycloalkenyl” as described herein. Examples include cyclopropenylsulfinyl, cyclobutenylsulfinyl, cyclopentenylsulfinyl, cyclohexenylsulfinyl, cycloheptenylsulfinyl, and cyclooctenylsulfinyl. C3-C6 cycloalkenylsulfinyl is preferred.

The term “cycloalkenylsulfonyl” includes a group in which sulfonyl is substituted with one “cycloalkenyl” as described herein. Examples include cyclopropenylsulfonyl, cyclobutenylsulfonyl, cyclopentenylsulfonyl, cyclohexenylsulfonyl, cycloheptenylsulfonyl, and cyclooctenylsulfonyl. Preferably C3-C6 cycloalkenylsulfonyl is preferred.

The term “cycloalkenylsulfonyloxy” includes a group in which an oxygen atom is substituted with one “cycloalkenylsulfonyl” described as described herein. Examples include cyclopropenylsulfonyloxy, cyclobutenylsulfonyloxy, cyclopentenylsulfonyloxy, cyclohexenylsulfonyloxy, cycloheptenylsulfonyloxy, and cyclooctenylsulfonyloxy. C3-C6 cycloalkenylsulfonyloxy is preferred.

The term “alkyloxycarbonyl” includes a group in which carbonyl is substituted with one “alkyloxy” as described herein. Examples include methyloxycarbonyl, ethyloxycarbonyl, n-propyloxycarbonyl, isopropyloxycarbonyl, n-butyloxycarbonyl, tert-butyloxycarbonyl, and n-pentyloxycarbonyl. C1-C6, C1-C4, or C1-C3 alkyloxycarbonyl is preferred. C1-C2 alkyloxycarbonyl is further preferred.

The term “alkenyloxycarbonyl” includes a group in which carbonyl is substituted with one “alkenyloxy” as described herein. Examples include vinyloxycarbonyl, allyloxycarbonyl, 1-propenyloxycarbonyl, 2-butenyloxycarbonyl, and 2-pentenyloxyarbonyl. C2-C6, C2-C4, or C2-C3 alkyloxycarbonyl is preferred.

The term “alkynyloxycarbonyl” includes a group in which carbonyl is substituted with one “alkynyloxy” as described herein. Examples include ethynyloxycarbonyl, 1-propynyloxycarbonyl, 2-propynyloxycarbonyl, 2-butynyloxyarbonyl, and 2-pentynyloxycarbonyl. C2-C6, C2-C4, or C2-C3 alkynyloxycarbonyl is preferred.

The term “acyl” includes alkylcarbonyl wherein the part of alkyl is “alkyl” as described herein, alkenylcarbonyl wherein the part of alkenyl is “alkenyl” as described herein, alkynylcarbonyl wherein the part of alkynyl is “alkynyl” as described herein, cycloalkylcarbonyl wherein the part of cycloalkyl is “cycloalkyl” as described herein, arylcarbonyl wherein the part of aryl is “aryl” as described herein, heteroarylcarbonyl wherein the part of heteroaryl is “heteroaryl” as described herein, and non-aromatic heterocycliccarbonyl wherein the part of non-aromatic heterocyclic group is “non-aromatic heterocyclic group” as described herein. “Alkyl,” “alkenyl,” “alkynyl,” “cycloalkyl,” “aryl,” “heteroaryl,” and “non-aromatic heterocyclic group” may be substituted respectively with substituent groups exemplified in “optionally substituted alkyl,” “optionally substituted alkenyl,” “optionally substituted alkynyl,” “optionally substituted cycloalkyl,” “optionally substituted aryl,” “optionally substituted heteroaryl,” and “optionally substituted non-aromatic heterocyclic group” as described herein. Examples of the acyl group include acetyl, propionyl, butyroyl, cyclohexylcarbonyl, benzoyl, pyridinecarbonyl, and the like.

The term “optionally substituted amino” includes an amino group which may be substituted with one or two group(s) of “alkyl” as described herein, “alkenyl” as described herein, “alkynyl” as described herein, “cycloalkyl” as described herein, “cycloalkynyl” as described herein, “aryl” as described herein, “heteroaryl” as described herein, “acyl” as described herein, “alkyloxycarbonyl” as described herein, “alkenyloxycarbonyl” as described herein, “alkynyloxycarbonyl” as described herein, “alkyl sulfonyl,” “alkenylsulfonyl,” “alkynylsulfonyl,” “arylsulfonyl,” and/or “heteroarylsulfonyl” as described herein. Examples of the optionally substituted amino group include amino, methylamino, dimethylamino, ethylamino, diethylamino, ethylmethylamino, benzylamino, acetylamino, benzoylamino, methyloxycarbonylamino, and methanesulfonylamino. Amino, methylamino, dimethylamino, ethylmethylamino, diethylamino, acetylamino, and methanesulfonylamino are preferred.

The term “optionally substituted carbamoyl” includes an aminocarbonyl group wherein the part of optionally substituted amino is “optionally substituted amino” as described herein. Examples of the optionally substituted carbamoyl group includes carbamoyl, N-methylcarbamoyl, N,N-dimethylcarbamoyl, N-ethyl-N-methylcarbamoyl, N,N-diethylcarbamoyl, N-phenylcarbamoyl, N-benzylcarbamoyl, N-acetylcarbamoyl, and N-methylsulfonylcarbamoyl etc. Carbamoyl, N-methylcarbamoyl, N,N-dimethylcarbamoyl, and N-methylsulfonylcarbamoyl etc. are preferred.

The term “optionally substituted sulfamoyl” includes an aminosulfonyl group wherein the part of optionally substituted amino is “optionally substituted amino” as described herein. Examples of the optionally substituted sulfamoyl group include sulfamoyl, N-methylsulfamoyl, N,N-dimethylsulfamoyl, N-ethyl-N-methyl sulfamoyl, N,N-diethylsulfamoyl, N-phenylsulfamoyl, N-benzylsulfamoyl, N-acetylsulfamoyl, and N-methylsulfonylsulfamoyl etc. Sulfamoyl, N-methylsulfamoyl, N,N-dimethylsulfamoyl, and N-methylsulfonylsulfamoyl etc. are preferred.

The term “alkylene” means a straight or branched alkylene group having one to eight carbon atom(s). Examples include methylene, ethylene, 1-methylethylene, trimethylene, 1-methyltrimethylene, pentamethylene, hexamethylene, and the like. C1-C4 or C1-3 alkylenes are preferred. C1-C2 or C1 alkylene is further preferred.

The term “aryl” includes an aromatic monocyclic or aromatic fused cyclic hydrocarbons. It may be fused with “cycloalkyl” as described herein, “cycloalkenyl” as described herein or “non-aromatic heterocyclic group” as described herein at any possible position. Both of monocyclic ring and fused ring may be substituted at any position. Examples include phenyl, 1-naphthyl, 2-naphthyl, anthryl, tetrahydronaphthyl, 1,3-benzodioxolyl, 1,4-benzodioxanyl etc. Phenyl, 1-naphthyl, and 2-naphthyl are preferred. Phenyl is further preferred.

The term “non-aromatic heterocyclic group” includes a 5- to 7-membered non-aromatic heterocyclic ring containing one or more of heteroatom(s) selected independently from oxygen, sulfur, and nitrogen atoms or a multicyclic ring formed by fusing the two or more rings thereof. Examples include pyrrolidinyl (e.g., 1-pyrrolidinyl, 2-pyrrolidinyl), pyrrolinyl (e.g., 3-pyrrolinyl), imidazolidinyl (e.g., 2-imidazolidinyl), imidazolinyl (e.g., imidazolinyl), pyrazolidinyl (e.g., 1-pyrazolidinyl, 2-pyrazolidinyl), pyrazolinyl (e.g., pyrazolinyl), piperidyl (e.g., piperidino, 2-piperidyl), piperazinyl (e.g., 1-piperazinyl), indolinyl (e.g., 1-indolinyl), isoindolinyl (e.g., isoindolinyl), morpholinyl (e.g., morpholino, 3-morpholinyl) etc.

The term “heteroaryl” includes a 5- to 6-membered aromatic ring containing one or more of heteroatom(s) selected independently from oxygen, sulfur, and nitrogen atoms. It may be fused with “cycloalkyl” as described herein, “aryl” as described herein, “non-aromatic heterocyclic group” as described herein, or other heteroaryl at any possible position. The heteroaryl group may be substituted at any position whenever it is a monocyclic ring or a fused ring. Examples include pyrrolyl (e.g., 1-pyrrolyl, 2-pyrrolyl, 3-pyrrolyl), furyl (e.g., 2-furyl, 3-furyl), thienyl (e.g., 2-thienyl, 3-thienyl), imidazolyl (e.g., 2-imidazolyl, 4-imidazolyl), pyrazolyl (e.g., 1-pyrazolyl, 3-pyrazolyl), isothiazolyl (e.g., 3-isothiazolyl), isoxazolyl (e.g., 3-isoxazolyl), oxazolyl (e.g., 2-oxazolyl), thiazolyl (e.g., 2-thiazolyl), pyridyl (e.g., 2-pyridyl, 3-pyridyl, 4-pyridyl), pyrazinyl (e.g., 2-pyrazinyl), pyrimidinyl (e.g., 2-pyrimidinyl, 4-pyrimidinyl), pyridazinyl (e.g., 3-pyridazinyl), tetrazolyl (e.g., 1H-tetrazolyl), oxadiazolyl (e.g., 1,3,4-oxadiazolyl), thiadiazolyl (e.g., 1,3,4-thiadiazolyl), indolidinyl (e.g., 2-indolidinyl, 6-indolidinyl), isoindolynyl (e.g., 2-isoindolynyl), indolyl (e.g., 1-indolyl, 2-indolyl, 3-indolyl), indazolyl (e.g., 3-indazolyl), purinyl (e.g., 8-purinyl), quinolidinyl (e.g., 2-quinolidinyl), isoquinolyl (e.g., 3-isoquinolyl), quinolyl (e.g., 2-quinolyl, 5-quinolyl), phtharazinyl (e.g., 1-phtharazinyl), naphthylidinyl (e.g., 2-naphthylidinyl), quinolanyl (e.g., 2-quinolanyl), quinazolinyl (e.g., 2-quinazolinyl), cinnolinyl (e.g., 3-cinnolinyl), pteridinyl (e.g., 2-pteridinyl), carbazolyl (e.g., 2-carbazolyl, 4-carbazolyl), phenanthridinyl (e.g., 2-phenanthridinyl, 3-phenanthridinyl), acridinyl (e.g., 1-acridinyl, 2-acridinyl), dibenzofuranyl (e.g., 1-dibenzofuranyl, 2-dibenzofuranyl), benzoimidazolyl (e.g., 2-benzoimidazolyl), benzoisoxazolyl (e.g., 3-benzoisoxazolyl), benzooxazolyl (e.g., 2-benzooxazolyl), benzooxadiazolyl (e.g., 4-benzooxadiazolyl), benzoisothiazolyl (e.g., 3-benzoisothiazolyl), benzothiazolyl (e.g., 2-benzothiazolyl), benzofuryl (e.g., 3-benzofuryl), benzothienyl (e.g., 2-benzothienyl), dibenzothienyl (e.g., 2-dibenzothienyl), and benzodioxolyl (e.g., 1,3-benzodioxolyl), etc.

The term “aryloxy” includes a group in which an oxygen atom is substituted with one “aryl” as described herein. Examples include phenyloxy and naphthyloxy, etc.

The term “arylthio” includes a group in which a sulfur atom is substituted with one “aryl” as described herein. Examples include phenylthio and naphthylthio, etc.

The term “arylsulfinyl” includes a group in which sulfinyl is substituted with one “aryl” as described herein. Examples include phenylsulfinyl and naphthylsulfinyl, etc.

The term “arylsulfonyl” includes a group in which sulfonyl is substituted with one “aryl” as described herein. Examples include phenylsulfonyl and naphthylsulfoinyl, etc.

Examples of “arylsulfonyloxy” include phenylsulfonyloxy and naphthylsulfonyloxy, etc.

The term “aryloxycarbonyl” includes a group in which carbonyl is substituted with one “aryloxy” as described herein. Examples include phenyloxycarbonyl, 1-naphthyloxycarbonyl and 2-naphthyloxycarbonyl, etc.

The term “heteroaryloxy” includes a group in which an oxygen atom is substituted with one “heteroaryl” as described herein. Examples include pyrrolyloxy, furyloxy, thienyloxy, imidazolyloxy, pyrazolyloxy, isothiazolyloxy, isoxazolyloxy, oxazolyloxy, thiazolyloxy, pyridyloxy, pyrazinyloxy, pyrimidinyloxy, pyridazinyloxy, tetrazolyloxy, oxadiazolyloxy, thiadiazolyloxy, indolidinyloxy, isoindolynyloxy, indolyloxy, indazolyloxy, purinyloxy, quinolidinyloxy, isoquinolyloxy, quinolyloxy, phtharazinyloxy, naphthylidinyloxy, quinolanyloxy, quinazolinyloxy, cinnolinyloxy, pteridinyloxy, carbazolyloxy, phenanthridinyloxy, acridinyloxy, dibenzofuranyloxy, benzoimidazolyloxy, benzoisoxazolyloxy, benzooxazolyloxy, benzooxadiazolyloxy, benzoisothiazolyloxy, benzothiazolyloxy, benzofuryloxy, benzothienyloxy, dibenzothienyloxy, and benzodioxolyloxy. Preferably furyloxy, thienyloxy, imidazolyloxy, pyrazolyloxy, isothiazolyloxy, isoxazolyloxy, oxazolyloxy, thiazolyloxy, pyridyloxy, pyrazinyloxy, pyrimidinyloxy, and pyridazinyloxy, etc.

The term “heteroarylthio” includes a group in which a sulfur atom is substituted with one “heteroaryl” as described herein. Examples include pyrrolylthio, furylthio, thienylthio, imidazolylthio, pyrazolylthio, isothiazolylthio, isoxazolylthio, oxazolylthio, thiazolylthio, pyridylthio, pyrazinylthio, pyrimidinylthio, pyridazinylthio, tetrazolylthio, oxadiazolylthio, thiadiazolylthio, indolidinylthio, isoindolynylthio, indolylthio, indazolylthio, purinylthio, quinolidinylthio, isoquinolylthio, quinolylthio, phtharazinylthio, naphthylidinylthio, quinolanylthio, quinazolinylthio, cinnolinylthio, pteridinylthio, carbazolylthio, phenanthridinylthio, acridinylthio, dibenzofuranylthio, benzoimidazolylthio, benzoisoxazolylthio, benzooxazolylthio, benzooxadiazolylthio, benzoisothiazolylthio, benzothiazolylthio, benzofurylthio, benzothienylthio, dibenzothienylthio, and benzodioxolylthio, etc. Preferably furylthio, thienylthio, imidazolylthio, pyrazolylthio, isothiazolylthio, isoxazolylthio, oxazolylthio, thiazolylthio, pyridylthio, pyrazinylthio, pyrimidinylthio, and pyridazinylthio, etc.

The term “heteroarylsulfinyl” includes a group in which sulfinyl is substituted with one “heteroaryl” as described herein. Examples include pyrrolylsulfinyl, furylsulfinyl, thienylsulfinyl, imidazolylsulfinyl, pyrazolylsulfinyl, isothiazolylsulfinyl, isoxazolylsulfinyl, oxazolylsulfinyl, thiazolylsulfinyl, pyridylsulfinyl, pyrazinylsulfinyl, pyrimidinylsulfinyl, pyridazinylsulfinyl, tetrazolylsulfinyl, oxadiazolylsulfinyl, thiadiazolylsulfinyl, indolidinylsulfinyl, isoindolylsulfinyl, indolylsulfinyl, indazolylsulfinyl, purinylsulfinyl, quinolidinylsulfinyl, isoquinolylsulfinyl, quinolylsulfinyl, phtharazinylsulfinyl, naphthylidinylsulfinyl, quinolanylsulfinyl, quinazolinylsulfinyl, cinnolinylsulfinyl, pteridinylsulfinyl, carbazolylsulfinyl, phenanthridinylsulfinyl, acridinylsulfinyl, dibenzofuranylsulfinyl, benzoimidazolylsulfinyl, benzoisoxazolylsulfinyl, benzooxazolylsulfinyl, benzooxadiazolylsulfinyl, benzoisothiazolylsulfinyl, benzothiazolylsulfinyl, benzofurylsulfinyl, benzothienylsulfinyl, dibenzothienylsulfinyl, and benzodioxolylsulfinyl. Furylsulfinyl, thienylsulfinyl, imidazolylsulfinyl, pyrazolylsulfinyl, isothiazolylsulfinyl, isoxazolylsulfinyl, oxazolylsulfinyl, thiazolylsulfinyl, pyridylsulfinyl, pyrazinylsulfinyl, pyrimidinylsulfinyl, and pyridazinylsulfinyl are preferred.

The term “heteroarylsulfonyl” includes a group in which sulfonyl is substituted with one “heteroaryl” as described herein. Examples include pyrrolylsulfonyl, furylsulfonyl, thienylsulfonyl, imidazolylsulfonyl, pyrazolylsulfonyl, isothiazolylsulfonyl, isoxazolylsulfonyl, oxazolylsulfonyl, thiazolylsulfonyl, pyridylsulfonyl, pyrazinylsulfonyl, pyrimidinylsulfonyl, pyridazinylsulfonyl, tetrazolylsulfonyl, oxadiazolylsulfonyl, thiadiazolylsulfonyl, indolizinylsulfonyl, isoindolylsulfonyl, indolylsulfonyl, indazolylsulfonyl, purinylsulfonyl, quinolidinylsulfonyl, isoquinolylsulfonyl, quinolylsulfonyl, phtharazinylsulfonyl, naphthilidinylsulfonyl, quinolanylsulfonyl, quinazolinylsulfonyl, cinnolinyl sulfonyl, pteridinyl sulfonyl, carbazolylsulfonyl, phenanthridinylsulfonyl, acridinylsulfonyl, dibenzofuranylsulfonyl, benzoimidazolylsulfonyl, benzoisoxazolylsulfonyl, benzooxazolylsulfonyl, benzooxadiazolylsulfonyl, benzoisothiazolylsulfonyl, benzothiazolylsulfonyl, benzofurylsulfonyl, benzothienylsulfonyl, dibenzothienylsulfonyl, and benzodioxolylsulfonyl, etc. Furylsulfonyl, thienylsulfonyl, imidazolylsulfonyl, pyrazolylsulfonyl, isothiazolylsulfonyl, isoxazolylsulfonyl, oxazolylsulfonyl, thiazolylsulfonyl, pyridylsulfonyl, pyrazinylsulfonyl, pyrimidinylsulfonyl, and pyridazinylsulfonyl are preferred.

The term “heteroarylsulfonyloxy” includes a group in which an oxygen atom is substituted with one “heteroarylsulfonyl” as described herein. Examples include pyrrolylsulfonyloxy, furylsulfonyloxy, thienylsulfonyloxy, imidazolylsulfonyloxy, pyrazolylsulfonyloxy, isothiazolylsulfonyloxy, isoxazolylsulfonyloxy, oxazolylsulfonyloxy, thiazolylsulfonyloxy, pyridylsulfonyloxy, pyrazinylsulfonyloxy, pyrimidinylsulfonyloxy, pyridazinylsulfonyloxy, tetrazolylsulfonyloxy, oxadiazolylsulfonyloxy, thiadiazolylsulfonyloxy, indolizinylsulfonyloxy, isoindolylsulfonyloxy, indolylsulfonyloxy, indazolylsulfonyloxy, purinylsulfonyloxy, quinolidinylsulfonyloxy, isoquinolylsulfonyloxy, quinolylsulfonyloxy, phtharazinylsulfonyloxy, naphthilidinylsulfonyloxy, quinolanyl sulfonyloxy, quinazolinylsulfonyloxy, cinnolinylsulfonyloxy, pteridinylsulfonyloxy, carbazolylsulfonyloxy, phenanthridinylsulfonyloxy, acridinylsulfonyloxy, dibenzofuranylsulfonyloxy, benzoimidazolylsulfonyloxy, benzoisoxazolylsulfonyloxy, benzooxazolylsulfonyloxy, benzooxadiazolylsulfonyloxy, benzoisothiazolylsulfonyloxy, benzothiazolylsulfonyloxy, benzofuryl sulfonyloxy, benzothienylsulfonyloxy, dibenzothienylsulfonyloxy, and benzodioxolylsulfonyloxy, etc. Furylsulfonyloxy, thienylsulfonyloxy, imidazolylsulfonyloxy, pyrazolylsulfonyloxy, isothiazolylsulfonyloxy, isoxazolylsulfonyloxy, oxazolylsulfonyloxy, thiazolylsulfonyloxy, pyridylsulfonyloxy, pyrazinylsulfonyloxy, pyrimidinylsulfonyloxy, and pyridazinylsulfonyloxy are preferred.

The term “aromatic carbocyclic ring” includes an aromatic monocyclic or aromatic fused carbocyclic ring. Examples include a benzene ring, a naphthalene ring, and an anthracene ring. A benzene ring is preferred.

The term “aromatic heterocyclic ring” includes an aromatic monocyclic or aromatic fused heterocyclic ring. Examples include a pyrrole ring, a furan ring, a thiophen ring, a pyrazole ring, an imidazole ring, an isothiazole ring, an isoxazole ring, an oxazole ring, a thiazole ring, a pyrazine ring, a pyrimidine ring, a pyridazine ring, a tetrazole ring, an oxadiazole ring, a thiadiazole ring, an indolizine ring, an isoindole ring, an indole ring, an indazole ring, a purine ring, a quinolidine ring, an isoquinoline ring, a quinoline ring, a phtharazine ring, a naphthyridine ring, a quinolane ring, a quinazoline ring, a cinnoline ring, a pteridine ring, a carbazole ring, a phenanthridine ring, an acridine ring, a dibenzofuran ring, a benzimidazole ring, a benzisoxazole ring, a benzoxazole ring, a benzoxadiazole ring, a benzisothiazole ring, a benzothiazole ring, a benzofuran ring, a benzothiophene ring, a dibenzothiophene ring, and a benzodixolane ring are exemplified. Preferably a pyridine ring, a furan ring, and a thiophen ring are exemplified.

The term “C1-C6 alkylene” includes a straight or branched alkylene group having one to six carbon atom(s). Examples include —CH₂—, —CH(CH₃)—, —C(CH₃)₂—, —CH₂CH₂—, —CH(CH₃)CH₂—, —C(CH₃)₂CH₂—, —CH₂CH₂CH₂—, —CH₂CH₂CH₂CH₂—, —CH₂CH₂CH₂CH₂CH₂—, and —CH₂CH₂CH₂CH₂CH₂CH₂—. Preferred are —CH₂—, —CH₂CH₂—, —CH₂CH₂CH₂—, and —CH₂CH₂CH₂CH₂—.

The term “alkylene optionally containing one or two heteroatom(s)” of “optionally substituted alkylene optionally containing one or two heteroatom(s)” includes a straight or branched alkylene group having one to six carbon atoms, optionally containing one or two heteroatom(s) which may be substituted with “alkyl” as described herein. Examples include —CH₂—, —CH(CH₃)—, —C(CH₃)₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —CH₂O—, —OCH₂—, —CH₂CH₂O—, —OCH₂CH₂—, —CH₂S—, —SCH₂—, —CH₂CH₂S—, —SCH₂CH₂—, —CH₂CH₂OCH₂CH₂—, —OCH₂CH₂O—, —OCH₂O—, —NHCH₂—, —N(CH₃)CH₂—, —N⁺(CH₃)₂CH₂—, —NHCH₂CH₂CH₂—, and —N(CH₃)CH₂CH₂CH₂—, etc. Preferred are —CH₂—, —CH₂CH₂—, —CH₂CH₂CH₂—, —CH₂CH₂CH₂CH₂—, —OCH₂CH₂O—, —OCH₂O—, and —N(CH₃)CH₂CH₂CH₂—.

The term “alkenylene optionally containing one or two heteroatom(s)” of “optionally substituted alkenylene optionally containing one or two heteroatom(s)” includes a straight or branched alkenylene group having two to six carbon atoms, optionally containing one or two heteroatom(s) which may be substituted with “alkyl” as described herein. Examples include —CH═CHCH═CH—, —CH═CHO—, —OCH═CH—, —CH═CHS—, —SCH═CH—, —CH═CHNH—, —NHCH═CH—, —CH═CH—CH═N—, and —N═CH—CH═CH—. Preferred are, —CH═CHCH═CH—, —CH═CHCH═N—, and N═CHCH═CH—.

The term “alkynylene optionally containing one or two heteroatom(s)” includes a straight or branched alkynylene group having two to six carbon atoms, optionally containing one or two heteroatom(s) which may be substituted with “alkyl” as described herein. Examples include —C≡CCH₂—, —CH₂C≡CCH₂—, —CH₂C≡CCH₂O—, —OCH₂C≡CH—, —CH₂C≡CCH₂S—, —SCH₂C≡CH—, —CH₂C≡CCH₂NH—, —NHCH₂C≡CH—, —CH₂C≡CCH₂N(CH₃)—, and —N(CH₃)CH₂C≡CH—. Especially, —CH₂C≡CCH₂—, and —OCH₂C≡CH— are preferred.

The term “3- to 8-membered nitrogen-containing non-aromatic heterocyclic ring” includes a ring of any of the formulas described as such in U.S. Pat. No. 8,143,285, which is incorporated herein by reference in its entirety.

The term “3- to 8-nitrogen-containing aromatic heterocyclic ring” includes a 3- to 8-membered aromatic heterocyclic ring containing one or more of nitrogen atom(s), and further optionally an oxygen atom and/or sulfur atom in the ring. Examples include pyrrolyl (e.g., 1-pyrrolyl, 2-pyrrolyl, 3-pyrrolyl), imidazolyl (e.g., 2-imidazolyl, 4-imidazolyl), pyrazolyl (e.g., 1-pyrazolyl, 3-pyrazolyl), isothiazolyl (e.g., 3-isothiazolyl), isoxazolyl (e.g., 3-isoxazolyl), oxazolyl (e.g., 2-oxazolyl), thiazolyl (e.g., 2-thiazolyl), pyridyl (e.g., 2-pyridyl, 3-pyridyl, 4-pyridyl), pyrazinyl (e.g., 2-pyrazinyl), pyrimidinyl (e.g., 2-pyrimidinyl, 4-pyrimidinyl), pyridazinyl (e.g., 3-pyridazinyl), tetrazolyl (e.g., 1H-tetrazolyl), oxadiazolyl (e.g., 1,3,4-oxadiazolyl), and thiadiazolyl (e.g., 1,3,4-thiadiazolyl).

The term “4- to 8-membered nitrogen-containing heterocyclic ring containing one or two nitrogen atom(s)” means a ring of any of the formulas described as such in U.S. Pat. No. 8,143,285, which is incorporated herein by reference in its entirety.

The term “oxo” refers to an ═O group.

“Optionally substituted” is used interchangeably herein with “substituted or unsubstituted.”

In the present specification, examples of substituents in “optionally substituted alkyl,” “optionally substituted alkyloxy,” “optionally substituted alkylthio,” “optionally substituted alkylsulfinyl,” “optionally substituted alkylsulfonyl,” “optionally substituted alkylsulfonyloxy,” and “the” include cycloalkyl, alkylene optionally containing one or two heteroatom(s), hydroxyl, oxo, alkyloxy optionally substituted with a substituent group A at one to three position(s), thiol, alkylthio, halogen, nitro, cyano, carboxyl, sulfino (—SO₂H), alkyloxycarbonyl, optionally substituted amino, optionally substituted carbamoyl, acyl, aryl (e.g., phenyl) optionally substituted with a substituent group B at one to three position(s), heteroaryl (e.g., pyridyl, furyl, thienyl, imidazolyl, oxazolyl, thiazolyl, pyrazolyl) optionally substituted with a substituent group C at one to three position(s), an optionally substituted non-aromatic heterocyclic ring group (e.g., morpholinyl, pyrrolidinyl, piperazinyl) which may be substituted with a substituent group C at one to three position(s), aryloxy (e.g., phenyloxy) optionally substituted with a substituent group B at one to three position(s), alkylsulfonyl, and the like. The above-referenced “optionally substituted” moieties can be substituted with one to three of the above-referenced substituent(s) at any possible position.

In the present specification, examples of substituents in “optionally substituted alkenyl,” “optionally substituted alkynyl,” “optionally substituted alkenyloxy,” “optionally substituted alkynyloxy,” “optionally substituted alkenylthio,” “optionally substituted alkynylthio,” “optionally substituted alkenyloxycarbonyl,” “optionally substituted alkynyloxycarbonyl,” “optionally substituted cycloalkyl,” “optionally substituted cycloalkenyl,” “optionally substituted cycloalkyloxy, “optionally substituted cycloalkenyloxy,” “optionally substituted cycloalkylthio,” “optionally substituted cycloalkenylthio,” “optionally substituted cycloalkylsulfinyl,” “optionally substituted cycloalkenylsulfinyl,” “optionally substituted cycloalkylsulfonyl,” “optionally substituted cycloalkenylsulfonyl,” “optionally substituted cycloalkylsulfonyloxy,” “optionally substituted cycloalkenylsulfonyloxy,” “optionally substituted alkenyloxycarbonyl,” “optionally substituted alkylene,” “optionally substituted C1-C6 alkylene,” “optionally substituted alkylene optionally containing one or two heteroatom(s),” “optionally substituted alkenylene,” “optionally substituted alkenylene optionally containing one or two heteroatom(s),” “optionally substituted alkynylene,” and “optionally substituted alkynylene optionally containing one or two heteroatom(s)” include alkyl (such as dialkyl) optionally substituted with a substituent group D at one to three position(s), cycloalkyl, hydroxyl, oxo, alkyloxy optionally substituted with a substituent group A at one to three position(s), thiol, alkylthio, halogen, nitro, cyano, carboxyl, sulfino, alkyloxycarbonyl, optionally substituted amino, optionally substituted carbamoyl, acyl, acyloxy, aryl (e.g., phenyl) optionally substituted with a substituent group B at one to three position(s), heteroaryl (e.g., pyridyl, furyl, thienyl, imidazolyl, oxazolyl, thiazolyl, pyrazolyl) optionally substituted with a substituent group C at one to three position(s), non-aromatic heterocyclic group (e.g., morpholinyl, pyrrolidinyl, piperazinyl) optionally substituted with a substituent group C at one to three position(s), aryloxy (e.g., phenyloxy) optionally substituted with a substituent group C at one to three position(s), alkylsulfonyl, and the like. The above-referenced “optionally substituted” moieties can be substituted with one or more of the above-referenced substituent(s) at any possible position.

In the present specification, examples of substituents in “optionally substituted aryl,” “optionally substituted phenoxy,” “optionally substituted aryloxy,” “optionally substituted phenylthio,” “optionally substituted arylthio,” “optionally substituted arylsulfinyl,” “optionally substituted arylsulfonyl,” “optionally substituted arylsulfonyloxy,” “optionally substituted heteroaryl,” “optionally substituted heteroaryloxy,” “optionally substituted heteroarylthio,” “optionally substituted heteroarylsulfinyl,” “optionally substituted heteroarylsulfonyl,” “optionally substituted heteroarylsulfonyloxy,” “optionally substituted non-aromatic heterocyclic group,” “optionally substituted C6 arene-1,4-diamine-N¹,N⁴-diyl,” and substituted C6 arene-1,4-diamine-N¹,N⁴-diyl,” include alkyl optionally substituted with a substituent group D at one to three position(s), cycloalkyl, alkenyl, alkynyl, hydroxyl, alkyloxy optionally substituted with a substituent group A at one to three position(s), aryloxy (e.g., phenoxy) optionally substituted with a substituent group B at one to three position(s), thiol, alkylthio, halogen, nitro, cyano, carboxyl, sulfino, alkyloxycarbonyl, acyl, alkylsulfonyl, optionally substituted amino, optionally substituted carbamoyl, aryl (e.g., phenyl) optionally substituted with a substituent group B at one to three position(s), heteroaryl (e.g., pyridyl, furyl, thienyl, imidazolyl, oxazolyl, thiazolyl, pyrazolyl) optionally substituted with a substituent group C at one to three position(s), non-aromatic heterocyclic group (e.g., morpholinyl, pyrrolidinyl, piperazinyl) optionally substituted with a substituent group C at one to three position(s), and the like. The above-referenced “optionally substituted” moieties can be substituted with one or more of the above-referenced substituent(s) at any possible position.

Substituent group A is comprised of halogen and phenyl optionally substituted with one to three substituent(s) selected from the Substituent group B.

Substituent group B is comprised of halogen, alkyl, alkyloxy, cyano, and nitro.

Substituent group C is comprised of halogen and alkyl.

Substituent group D is comprised of halogen and alkyloxy.

Unless otherwise defined, the term “substituted” refers to a moiety comprising any substituent described herein.

The compound of the invention can be topically administered to the surface tissue within a topical formulation. The topical formulation may be in the form of an ointment, a gel, a cream, a patch, an eye ointment, a lotion, a liquid, an emulsion, a spray, a solution, a suspension, and the like.

In some embodiments, the compound may be present in the topical formulation in an amount of about 0.001% or less to about 10% or more by weight. In some embodiments, the compound may be present in the topical formulation in an amount of about 0.001% to about 5%, about 0.001% to about 1%, about 0.001% to about 0.5%, about 0.01% to about 0.5%, about 0.05% to about 0.5%, about 0.1% to about 1%, about 0.4% to about 0.6%, about 0.3% to about 0.7%, about 0.2% to about 0.9%, about 0.1% to about 0.5%, about 0.2% to about 0.5% by weight, or a value within any of these ranges. In some embodiments, the compound may be present in the topical formulation in an amount of about 0.001%, about 0.01%, about 0.03%, about 0.05%, about 0.1%, about 0.2%, about 0.25%, about 0.3%, about 0.4%, about 0.5%, about 0.6%, about 0.7%, about 0.75%, about 0.8%, about 0.9%, about 1.0%, about 3.0%, or about 5% by weight, or a range of any two of these values.

In addition to the compound, the topical formulation can comprise a solvent, a base, an absorption enhancer, a bleeding preventing agent, water, a coloring agent, a flavoring agent, a preservative, an antioxidant, a stabilizer, an excipient, a carrier, a diluent, a usability improving agent, and/or any other suitable topical formulation ingredient.

Any solvent commonly used for a topical formulation may be used, including, but not limited to, polyethylene glycol having an average molecular weight of 200 to 600, dipropylene glycol, benzyl alcohol, polyoxyethylene sorbitan fatty acid ester, diethylene glycol monoethyl ether, propylene glycol, polyoxyethylene oleyl ether, polyoxyethylene octyl phenyl ether, polyoxyethylene lauryl ether, polyoxyethylene castor oil, oleic acid or a combination thereof. In some embodiments, the solvent comprises polyethylene glycol having an average molecular weight of 200 to 600. Suitable polyethylene glycols include, but are not limited to the Carbowax™ and Carbowax™ Sentry series (available from Dow), the Lipoxol™ series (available from Brenntag), the Lutrol™ series (available from BASF), and the Pluriol™ series (available from BASF). In some embodiments, the solvent may be in an amount of greater than about 30% by weight. In some embodiments, the solvent may be in an amount of greater than about 30% by weight to about 60% by weight. In some embodiments, the solvent may be in an amount of about 35% by weight to about 60%, about 35% to about 55%, about 35% to about 50%, about 40% to about 60%, or about 40% to about 55%, about 40% to about 50% by weight. In some embodiments, the solvent may be in an amount of about 35%, about 40%, about 45%, about 50%, about 55%, about 60%, or about 65% by weight, or a range of any two of these values.

The base may comprise any base commonly used in a topical formulation. Exemplary suitable bases include oleaginous bases, including, but not limited to, petrolatum, squalane, paraffin, liquid paraffin, microcrystalline wax, carnauba wax, white beeswax and the like. In some embodiments, the base comprises petrolatum. As used herein, petrolatum means a mixture of semi solid saturated hydrocarbons typically obtained from petroleum. In some embodiments, petrolatum comprises white petrolatum, mineral oil, petroleum jelly, yellow petrolatum, amber petrolatum, vasoliments, cosmoline, saxoline, stanoline, vasiline, cold tar, or a combination thereof. In some embodiments, the base is white petrolatum. In some embodiments, the base comprises U.S. Pharmacopoeia (USP) white petrolatum. In some embodiments, the base may be in an amount of less than about 10% by weight. In some embodiments, the base may be in an amount of about 0.001% to about 10% by weight. In some embodiments, the base may be in an amount of about 0.1% by weight to about 10%, about 0.5% to about 10%, about 1% to about 10%, about 2.5% to about 10%, or about 4% to about 10%, about 0.1% by weight to about 7%, about 0.5% to about 7%, about 1% to about 7%, about 2.5% to about 7%, or about 4% to about 7%, about 4% to about 5% by weight. In some embodiments, the base may be in an amount of about 0.1%, about 1%, about 2%, about 3%, about 4%, about 4.5%, about 5%, about 6%, about 7%, about 8%, about 9%, or about 10% by weight, or a range of any two of these values.

The absorption enhancer may comprise any absorption enhancer commonly used in a topical formulation. Exemplary suitable absorption enhancers include isopropyl myristate, ethyl myristate, octyldodecyl myristate, isopropyl palmitate, isostearyl palmitate, isopropyl isostearate, butyl stearate, ethyl oleate, decyl oleate, diisopropyl sebacate, diethyl sebacate, diisopropyl adipate, diethyl adipate, diethyl phthalate, or a combination thereof. It is believed that the skin absorbability of the compound can be further improved by adding an absorption enhancer. In some embodiments, the absorption enhancer may be in an amount of about 5% to about 20% by weight. In some embodiments, the absorption enhancer may be in an amount of about 5%, about 10%, about 15%, or about 20% by weight, or a range of any two of these values.

The bleeding preventing agent may comprise any bleeding agent commonly used in a topical formulation. Exemplary suitable bleeding preventing agents include polyethylene glycol having an average molecular weight of 1000 to 50000, polyoxyethylene hydrogenated castor oil, stearic acid, oleic acid, sorbitan monostearate, sorbitan monooleate, sorbitan sesquioleate, sorbitan trioleate, a glycerol ester of a fatty acid, other similar agents, or a combination thereof. In some embodiments, the glycerol ester of a fatty acid may comprise glycerol monostearate, diglyceryl isostearate, hexaglyceryl polyricinoleate, or other similar agents, or a combination thereof. In some embodiments, the glycerol ester of a fatty acid comprises glycerol monostearate. In some embodiments, the glycerol monostearate comprises glycerol monostearate 40-55 sold under the trademark Geleol™ Mono and Diglycerides NF. “Glycerol monostearate” is used interchangeably herein with “glyceryl monostearate.” It is believed that the bleeding of ingredients (in particular, a solvent and absorption enhancer) from a topical formulation can be prevented by adding a bleeding preventing agent, and thus improved stability can be achieved. In some embodiments, the total amount of the bleeding preventing agent may be in an amount of about 20% to about 50% by weight. In some embodiments, the total amount of the bleeding preventing agent may be in an amount of greater than about 25%. In some embodiments, the total amount of the bleeding preventing agent may be in an amount of about 25% to about 50% by weight, about 25% to about 40% by weight, about 25% to about 35% by weight, or about 30% to about 50% by weight. In some embodiments, the total amount of the bleeding preventing agent may be in an amount of about 25%, about 30%, about 33%, about 35%, about 40%, about 45%, or about 50% by weight, or a range of any two of these values.

The water may comprise purified water. In some embodiments, the water may be in an amount of about 0.1% to about 5% by weight. In some embodiments, the water may be in an amount of about 0.3% to about 4%, about 0.3% to about 3%, about 0.5% to about 3%, or about 0.5% to about 2%. In some embodiments, the water may be in an amount of about 0.1%, about 1%, about 2%, about 3%, about 4%, about 4.5%, or about 5% by weight, or a range of any two of these values.

The coloring agent may comprise any coloring agent commonly used in a topical formulation. Exemplary suitable coloring agents include iron sesquioxide, yellow iron sesquioxide, carmine, caramel, beta-carotene, titanium oxide, talc, riboflavin sodium phosphate, yellow aluminum lake, or a combination thereof.

The flavoring agent may comprise any flavoring agent commonly used in a topical formulation. Exemplary suitable flavoring agents include from cocoa powder, mentha oil, menthol, lemon oil, borneol, powdered cinnamon bark, ascorbic acid, citric acid, tartaric acid, malic acid, aspartame, potassium acesulfame, or a combination thereof.

The preservative may comprise any preservative commonly used in a topical formulation. Exemplary suitable preservatives include methylparaben, propylparaben, chlorobutanol, benzyl alcohol, phenethyl alcohol, dehydroacetic acid, sorbic acid or a combination thereof.

The antioxidant may comprise any antioxidant commonly used in a topical formulation. Exemplary suitable antioxidants include sulfite salts, ascorbic acid, tocopherol, lycopene, green tea, coffee berry, resveratrol, grape seed, niacinamide, genistein, ferulic acid, idebenone, coenzyme Q10, retinol, vitamin A, lutein, zeaxanthin, astaxanthin, alpha lipoic acid, zinc, copper, beta-carotene, or a combination thereof.

The stabilizer may comprise any stabilizer commonly used in a topical formulation. Exemplary suitable stabilizers include ascorbic acid, edetic acid salt, erythorbic acid, tocopherol, and the like, or a combination thereof.

The usability improving agent may comprise any usability improving agent commonly used in a topical formulation. Exemplary suitable usability improving agents include polyoxyethylene hydrogenated castor oil 40, polyoxyethylene hydrogenated castor oil 60, and the like, or a combination thereof.

The carrier, diluent, or excipient may comprise any commonly used in a topical formulation. Common carrier, diluent, and excipients are described in standard reference works such as Goodman & Gilman's The Pharmacological Basis of Therapeutics. 11th ed. New York: McGraw-Hill; 2005, which is hereby incorporated by reference in its entirety. Exemplary carriers, diluents, or excipients include lauryl lactate (an emollient/permeation enhancer), diethylene glycol monoethylether (an emollient/permeation enhancer), DMSO (a solubility enhancer), silicone elastomer (a rheology/texture modifier), caprylic/capric triglyceride, (an emollient), octisalate, (an emollient/UV filter), silicone fluid (an emollient/diluent), squalene (an emollient), sunflower oil (an emollient), and silicone dioxide (a thickening agent).

Exemplary formulations of the invention comprise about 1-5% w/w of a compound of the invention and one or more of propylene glycol as a humectant/vehicle in an amount from about 20% w/w to about 27% w/w, white petrolatum as an emollient/thickener in an amount from about 10% w/w to about 12% w/w, stearyl alcohol as an emulsion stabilizer/thickener in an amount from about 10% w/w to about 12% w/w, glyceryl monostearate as an emulsifying agent/thickener in an amount from about 2.5% w/w to about 5% w/w, Span 20 as an emulsifying agent in an amount of about 0.05% w/w, tween 80 as an emulsifying agent in an amount of about 4.5% w/w, and water in an amount from about 42.5% w/w to about 48% w/w.

In some embodiments, the topical formulation may be administered once daily, twice daily, bi-weekly, weekly, three times a week, every two weeks, every other day, monthly, every two months, every three months or as directed by a packaging or a physician to achieve the desired clinical result.

The amount of the compound to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician).

The term “about” means plus or minus 10% of the numerical value of the number with which it is being used. Therefore, about 50% means in the range of 45%-55%.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### Example 1. Use of Topical BT-11 Dihydrochloride Salt in a Model of Psoriasis

### Introduction

Psoriasis afflicts over 7 million people in the United States and 15 million worldwide, with over 95 million worldwide afflicted by inflammatory skin diseases, inclusive of psoriasis, atopic dermatitis, and rosacea. The resultant itchiness, effects on appearance, and persistent rashes have a significant impact on quality of life. In psoriasis, the most successful therapies have targeted Th17 cells and the IL-17/IL-23 axis. As such, demonstrating the ability of novel therapeutics to impact the differentiation of these cells, in vivo, is a critical mechanistic finding. Importantly, impacting Th17/Treg plasticity may indicate a mechanistic avenue for the maintenance of clinical responses by establishing a tolerogenic environment. Meanwhile, Th17 cells are believed to be the most responsive to metabolic manipulation, suggesting a susceptibility to the immunometabolic effects of the LANCL2 pathway.

### Methods

Imiquimod (IMQ)-induced model. C57BL/6 mice were anesthetized, shaved, and briefly exposed to depilatory cream on the surface of the back. Mice were given three days to recover from the procedure prior to entry to the study. After 3 days, mice were challenged with approximately 60 mg of 0.5% imiquimod cream daily by spreading cream over the shaved area. Treatment with BT-11 dihydrochloride salt (200 μg) in dimethyl sulfoxide (DMSO) or a DMSO control lacking BT-11 occurred by topical application to the shaved area. Mice were scored for erythema, scaling and skin thickness, summarized into a composite psoriasis area and severity index (PASI) score on a scale from 0 to 12, on day 7.

Analysis. Spleens were excised and crushed by microscope slides. Red blood cells were hypotonically lysed from the resultant suspension. Samples were filtered, washed and centrifuged prior to staining. Production of TNF, IL-17, and IL-21 were quantified from CD3+CD4+ T cells and IL6 from CD45+ by flow cytometry.

### Results

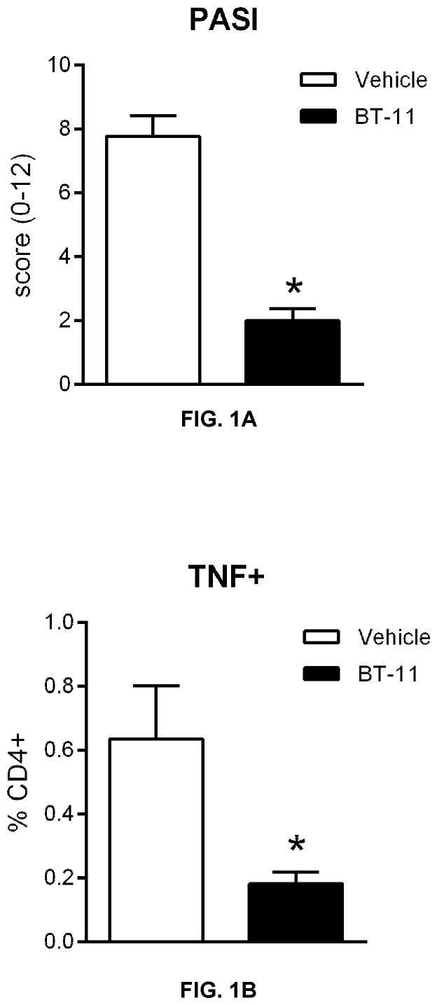

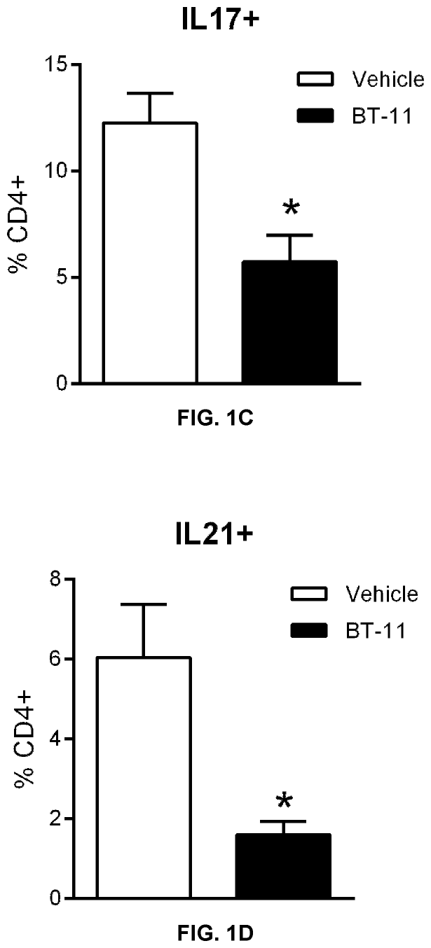

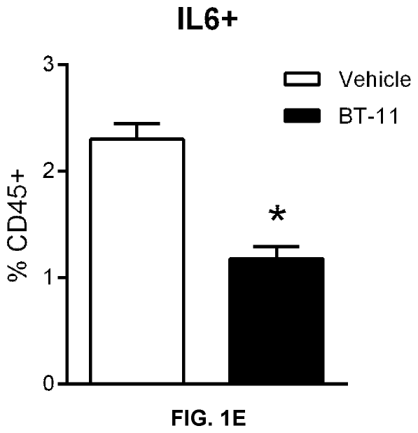

Topical BT-11 reduced severity of disease (FIG. (A) by approximately 750 on average. Reductions in overall PASI resulted from reductions in each subscore (erythema, scaling, skin thickness), indicating an overall improvement in disease severity. In the spleen, BT-11 reduced the proportions of TNF (FIG. 13), IL-17 (FIG. 1C) and IL-21 (FIG. 1D) positive CD4+ T cells, suggesting the potential to improve psoriasis-associated inflammation. Further, reduction in the proportions of IL-6+ cells (FIG. 1E) was observed within all CD45+ immune cells.

### Example 2. Exemplary Topical BT-11 Dihydrochloride Cream

Topical cream formulations containing BT-11 dihydrochloride (Formulation 1 and Formulation 2) were generated. The formulations are shown in Tables 1 and 2. The characterization of the prepared creams is shown in Table 3.

TABLE 1
Exemplary topical BT-11 dihydrochloride cream formulations.

| | | Concentration (% w/w) | |
|---|---|---|---|
| Ingredient | Functionality | Formulation 1 | Formulation 2 |
| BT-11 | Active Ingredient | 3 | 3 |
| Propylene Glycol | Humectant/Vehicle | 20 | 27 |
| White Petrolatum | Emollient/Thickener | 12 | 10 |
| Stearyl Alcohol | Emulsion Stabilizer/ | 12 | 10 |
| | Thickener | | |
| Glyceryl | Emulsifying | 5 | 2.5 |
| Monostearate | Agent/Thickener | | |
| Span 20 | Emulsifying Agent | 0.5 | 0.5 |
| Tween 80 | Emulsifying Agent | 4.5 | 4.5 |
| Water | Carrier | 48 | 42.5 |
| | | 100 | 100 |

TABLE 2
Exemplary topical BT-11 dihydrochloride cream formulations.

| | | Conceitration (% w/w) | | | | | | | | |
|---|---|---|---|---|---|---|---|---|---|---|
| Ingredient | Functionality | F3 | F4 | F5 | F6 | F7 | F8 | F9 | F10 | F11 |
| BT-11 | Active Ingredient | 2 | 0.5 | 2 | 0.5 | 2 | 2 | 2 | 3 | 4 |
| Propylene Gycol | Humectant/Vehicle | 28 | 28.6 | 26 | 26.6 | 26 | 26 | 26 | 27 | 28.6 |
| White Petrolatum | Emollient/Thickener | 10 | 10 | 10 | 10 | 10 | 10 | 10 | 11 | 10 |
| Stearyl Alcohol | Emulsion Stabilizer/ | 10 | 10 | 10 | 10 | 10 | 10 | 10 | 9 | 10 |
| | Thickener | | | | | | | | | |
| Glyceryl Monostearate | Emulsifying Agent/ | 2.5 | 2.5 | 2 | 2 | 2 | 2 | 2 | 2.5 | 2.5 |
| | Thickener | | | | | | | | | |
| Span 20 | Emulsifying Agent | 0.5 | 0.5 | 0.5 | 0.5 | 0.5 | 0.5 | 0.5 | 0.5 | 0.5 |
| Tween 80 | Emulsifying Agent | 4.5 | 4.5 | 5 | 5 | 5 | 5 | 5 | 5 | 4.5 |
| HPMC | Emulsifying Agent/ | 0 | 0 | 1 | 1 | 0 | 0 | 0 | 0 | 0 |
| | Thickener | | | | | | | | | |
| Butylated Hydroxyanisole | Stabilizer | 0.2 | 0.2 | 0.2 | 0.2 | 0.2 | 0.2 | 0.2 | 0 | 0.2 |
| Disodium Edetate | Stabilizer | 1 | 1 | 1 | 1 | 1 | 1 | 1 | 0 | 1 |
| Benzyl Alcohol | Preservative | 0 | 0.9 | 0 | 0.9 | 0 | 0 | 0 | 0 | 0.9 |
| Sodium Hydroxide | pH | 0 | 0 | 0 | 0 | 0 | 0.4 | 0.1 | 0 | 0.2 |
| Water | Carrier | 41.3 | 41.3 | 42.3 | 42.3 | 43.3 | 42.9 | 43.2 | 42 | 37.6 |
| | | 100 | 100 | 100 | 100 | 100 | 100 | 100 | 100 | 100 |

TABLE 3
Characterization of exemplary topical BT-11 dihydrochloride cream formulations.

| | | Viscosity | | | |
|---|---|---|---|---|---|
| Formulation | Appearance | Replicate | Viscosity (cP) | % Torque | % Assay |
| Formulation 1 | White to off-white | 1 | 1421 | 75.8 | 103.0 |
| | colored cream | 2 | 1592 | 84.9 | |
| | | 3 | 1553 | 82.8 | |
| | | Average | 1522 | 81.2 | |
| Formulation 2 | White to off-white | 1 | 1249 | 66.6 | 103.2 |
| | colored cream | 2 | 1198 | 63.9 | |
| | | 3 | 1125 | 60.0 | |
| | | Average | 1191 | 63.5 | |

### Example 3. Use of Topical BT-11 Dihydrochloride Cream in a Model of Atopic Dermatitis

### Introduction

Atopic dermatitis affects 10 to 20% of children and 1 to 3% of adults, with over 95 million worldwide afflicted by inflammatory skin diseases, inclusive of psoriasis, atopic dermatitis, and rosacea. The resultant itchiness, effects on appearance, and persistent rashes have a significant impact on quality of life. In atopic dermatitis, the immunological pathogenesis is often attributed to type 2 immune responses, which are commonly associated with a variety of allergic diseases. Type 2 immunes responses are driven in part by increased production of IL-4 and IL-5 cytokines.

### Methods

MC903-induced model. MC903 (2 nM in 20 uL ethanol) was applied to the dorsal ear skin three times per week for nine applications. BT-11 topical cream (2%) (Formula F3 is Table 2) was applied to the ear daily. Mice were scored for erythema, scaling and skin thickness, summarized into a composite disease activity index score through day 19.

Analysis. Cervical lymph nodes were excised and crushed by microscope slides. Red blood cells were hypotonically lysed from the resultant suspension. Samples were filtered, washed and centrifuged prior to staining. Production of IFNγ, IL-5, and IL-4 were quantified from CD3+CD4+ T cells by flow cytometry.

### Results

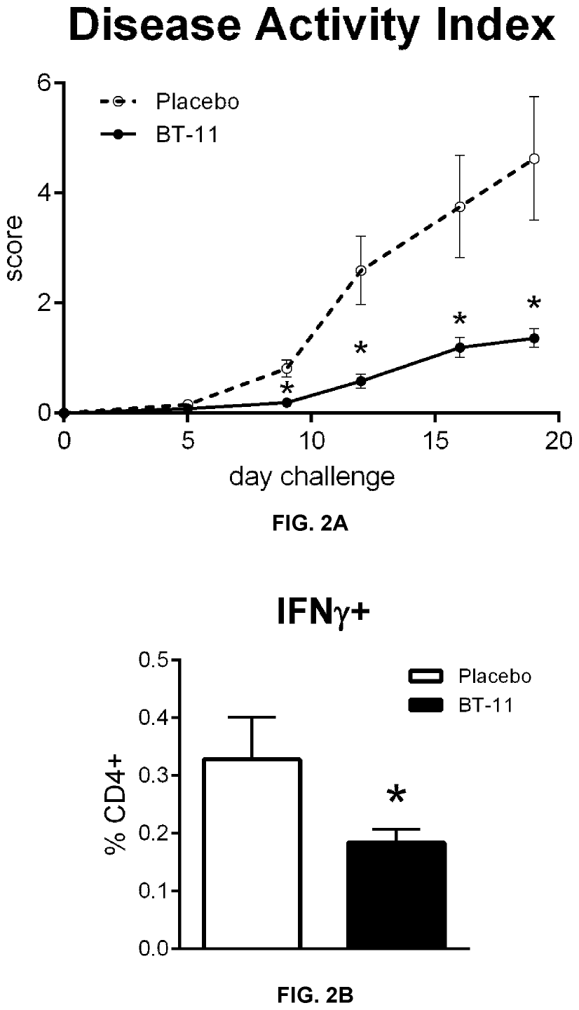

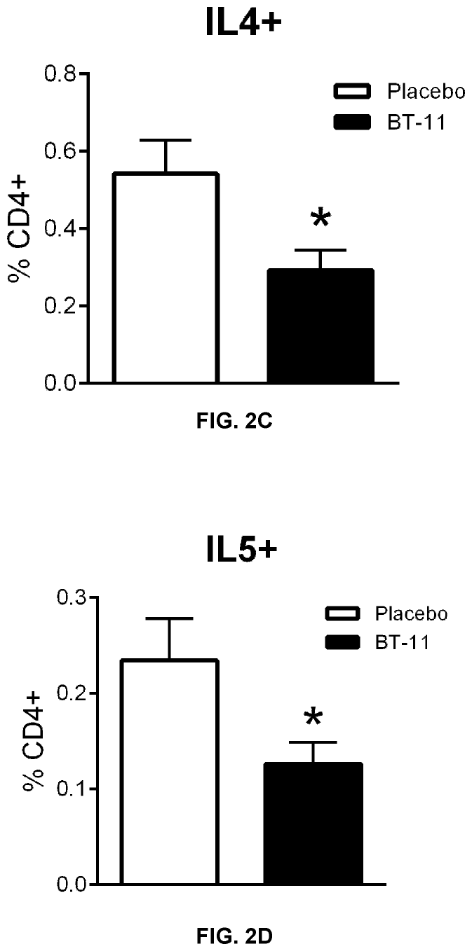

Topical BT-11 reduced severity of disease (FIG. 2A) by approximately 70% on average. Reductions in overall disease activity resulted from reductions in each subscore (erythema, scaling, skin thickness), indicating an overall improvement in disease severity. In the cervical lymph nodes, BT-11 reduced the proportions of IFNy (FIG. 2B), IL-4 (FIG. 2C) and IL-5 (FIG. 2D) positive CD4+ T cells, suggesting the potential to improve dermatitis-associated inflammation.

1. A method of treating an inflammatory or immune-mediated condition of a surface tissue, the method comprising topically administering to the surface tissue an amount of a compound effective to treat the condition, wherein the compound is a compound of formula (I): 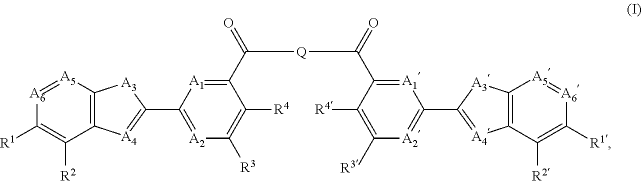
or a pharmaceutically acceptable salt thereof, wherein:
Q is piperazine-1,4-diyl; 2,5-diazabicyclo[2.2.1]heptane-2,5-diyl; 2,5-diazabicyclo[2.2.2]octane-2,5-diyl; 1,4-diazepane-1,4-diyl; benzene-1,4-diamine-N¹,N⁴-diyl; ethane-1,2-diamine-N¹,N²-diyl; N¹,N²-dialkylethane-1,2-diamine-N¹,N²-diyl; propane-1,3-diamine-N¹,N³-diyl; N¹,N³-dialkylpropane-1,3-diamine-N¹,N³-diyl; 1,4-diaminoanthracene-9,10-dione-1,4-diyl; C₆ arene-1,4-diamine-N¹,N⁴-diyl wherein the arene is substituted with one to four substituents independently selected from the group consisting of halogen, hydroxyl, carboxyl, optionally substituted alkyl, optionally substituted alkoxy, optionally substituted alkyloxycarbonyl, and optionally substituted aryl; or substituted piperazine-1,4-diyl wherein the piperazine is substituted with one to eight substituents independently selected from the group consisting of halogen, hydroxyl, carboxyl, optionally substituted alkyl, optionally substituted alkoxy, optionally substituted alkyloxycarbonyl, and optionally substituted aryl;
A₁ and A₁′ are each independently N or CR⁶;
A₂ and A₂′ are each independently N or CR⁷;
A₃ is NR⁸;
A₃′ is NR⁸, O, or S;
A₄ and A₄′ are each independently N or CR⁹;
A₅ and A₅′ are each independently N or CR¹⁰;
A₆ and A₆′ are each independently N or CR¹¹; and
R¹, R^{1′}, R², R^{2′}, R³, R^{3′}, R⁴, R^{4′}, R⁶, R⁷, R⁸, R⁹, R¹⁰, and R¹¹, if present, are in each instance independently hydrogen, halogen, optionally substituted alkyl, optionally substituted alkenyl, optionally substituted alkynyl, optionally substituted cycloalkyl, optionally substituted cycloalkenyl, hydroxyl, carboxyl, optionally substituted alkyloxy, optionally substituted alkenyloxy, optionally substituted alkynyloxy, optionally substituted cycloalkyloxy, optionally substituted cycloalkenyloxy, thiol, optionally substituted alkylthio, optionally substituted alkenylthio, optionally substituted alkynylthio, optionally substituted alkylsulfinyl, optionally substituted alkylsulfonyl, optionally substituted alkylsulfonyloxy, optionally substituted cycloalkylthio, optionally substituted cycloalkylsulfinyl, optionally substituted cycloalkylsulfonyl, optionally substituted cycloalkylsulfonyloxy, optionally substituted cycloalkenylthio, optionally substituted cycloalkenylsulfinyl, optionally substituted cycloalkenylsulfonyl, optionally substituted cycloalkenylsulfonyloxy, optionally substituted amino, acyl, optionally substituted alkyloxycarbonyl, optionally substituted alkenyloxycarbonyl, optionally substituted alkynyloxycarbonyl, optionally substituted aryloxycarbonyl, optionally substituted carbamoyl, optionally substituted sulfamoyl, cyano, nitro, optionally substituted aryl, optionally substituted aryloxy, optionally substituted arylthio, optionally substituted arylsulfinyl, optionally substituted arylsulfonyl, optionally substituted arylsulfonyloxy, optionally substituted heteroaryl, optionally substituted heteroaryloxy, optionally substituted heteroarylthio, optionally substituted heteroarylsulfinyl, optionally substituted heteroarylsulfonyl, optionally substituted heteroarylsulfonyloxy, or an optionally substituted non-aromatic heterocyclic group.

2. The method of claim 1, wherein the condition comprises at least one of psoriasis, cutaneous lupus erythematosus, dermatomyositis, pemphigoid, pemphigus, scleroderma, vasculitis, epidermolysis bullosa acquisita, vitiligo, lichen planus, scleritis, dermatitis, erythema nodosum, pyoderma gangrenosum, skin fissures, acne, enterocutaneous fistula, skin tags, canker sores, acrodermatitis enteropathica, pyoderma vegetans, leukocytoclastic vasculitis, anal fissures, Sweet's syndrome, rosacea, alopecia, keratoderma blennorrhagica, rosacea, cold sores, urticaria, actinic keratosis, carbuncle, cellulitis, ichthyosis vulgaris, skin infection, malar rash, photosensitivity, livedo reticularis, livedo reticularis, oral and nasal ulcers, purpura, mucositis, hemorrhoids, burn, and sunburn.

3. The method of claim 1, wherein the condition comprises an inflammatory or immune-mediated condition of skin, the surface tissue comprises the skin, and the method comprises topically administering the compound to the skin in an amount effective to treat the inflammatory or immune-mediated condition of the skin.

4. The method of claim 3, wherein the inflammatory or immune-mediated condition of the skin comprises at least one of psoriasis, cutaneous lupus erythematosus, dermatomyositis, pemphigoid, pemphigus, scleroderma, vasculitis, epidermolysis bullosa acquisita, vitiligo, lichen planus, scleritis, dermatitis, erythema nodosum, pyoderma gangrenosum, skin fissures, acne, enterocutaneous fistula, skin tags, acrodermatitis enteropathica, pyoderma vegetans, leukocytoclastic vasculitis, anal fissures, Sweet's syndrome, rosacea, alopecia, keratoderma blennorrhagica, rosacea, cold sores, urticaria, actinic keratosis, carbuncle, cellulitis, ichthyosis vulgaris, skin infection, malar rash, photosensitivity, livedo reticularis, livedo reticularis, purpura, hemorrhoids, burn, and sunburn.

5. The method of claim 1, wherein the condition comprises an inflammatory or immune-mediated condition of mucosa, the surface tissue comprises the mucosa, and the method comprises topically administering the compound to the mucosa in an amount effective to treat the inflammatory or immune-mediated condition of the mucosa.

6. The method of claim 5, wherein the inflammatory or immune-mediated condition of the mucosa comprises at least one of cutaneous lupus erythematosus, dermatomyositis, pemphigoid, pemphigus, scleroderma, epidermolysis bullosa acquisita, lichen planus, pyoderma gangrenosum, canker sores, acrodermatitis enteropathica, pyoderma vegetans, anal fissures, Sweet's syndrome, cold sores, oral and nasal ulcers, mucositis, hemorrhoids, and burn.

7. The method of claim 1, wherein the condition comprises an inflammatory or immune-mediated condition of the oral mucosa, the surface tissue comprises the oral mucosa, and the method comprises topically administering the compound to the oral mucosa in an amount effective to treat the inflammatory or immune-mediated condition of the oral mucosa.

8. The method of claim 7, wherein the inflammatory or immune-mediated condition of the oral mucosa comprises at least one of cutaneous lupus erythematosus, dermatomyositis, pemphigoid, pemphigus, scleroderma, epidermolysis bullosa acquisita, lichen planus, pyoderma gangrenosum, canker sores, acrodermatitis enteropathica, pyoderma vegetans, Sweet's syndrome, cold sores, oral ulcers, oral mucositis, and burn.

9. The method of claim 1, comprising wherein the condition comprises an autoimmune condition.

10. The method of claim 9, wherein the autoimmune condition comprises at least one of psoriasis, cutaneous lupus erythematosus, dermatomyositis, pemphigoid, pemphigus, scleroderma, vasculitis, epidermolysis bullosa acquisita, vitiligo, lichen planus, scleritis, dermatitis, erythema nodosum, pyoderma gangrenosum, skin fissures, acne, enterocutaneous fistula, skin tags, canker sores, acrodermatitis enteropathica, pyoderma vegetans, leukocytoclastic vasculitis, anal fissures, Sweet's syndrome, rosacea, and alopecia areata.

11. The method of claim 1, comprising topically administering the compound to the surface tissue of a subject suffering from an autoimmune disease.

12. The method of claim 11, wherein the autoimmune disease comprises at least one of inflammatory bowel disease, celiac disease, systemic lupus erythematosus, rheumatoid arthritis, type 1 diabetes, psoriatic arthritis, and Sjögren syndrome.

13. The method of claim 1, wherein the condition comprises at least one of psoriasis and atopic dermatitis.

14. The method of claim 1, wherein Q is piperazine-1,4-diyl or substituted piperazine-1,4-diyl wherein the piperazine in the substituted piperazine-1,4-diyl is substituted with one to eight substituents independently selected from the group consisting of (C₁ to C₆)alkyl, aryl, aryl(C₁ to C₆)alkyl, C(O)OH, and C(O)O(C₁ to C₆)alkyl.

15. The method of claim 1, wherein Q is piperazine-1,4-diyl.

16. The method of claim 15, wherein:
A₂ and A₂′ are each CR⁷;
A₃′ is NR⁸ or O;
A₄ and A₄′ are each N;
A₅ and A₅′ are each CR¹⁰; and
A₆ and A₆′ are each CR¹¹.

17. The method of claim 16, wherein A¹ and A₁′ are each N.

18-20. (canceled)

21. The method of claim 16, wherein A₃′ is NR⁸.

22-27. (canceled)

28. The method of claim 16, wherein R¹, R^{1′}, R², R^{2′}, R³, R^{3′}, R⁴, R^{4′}, R⁶, R⁷, R⁸, R¹⁰, and R¹¹, if present, are in each instance independently hydrogen, halogen, optionally substituted C1-C6 alkyl, hydroxyl, carboxyl, optionally substituted cycloalkyl, optionally substituted C1-C6 alkyloxy, optionally substituted amino, acyl, optionally substituted alkyloxycarbonyl, optionally substituted aryl, optionally substituted heteroaryl, or optionally substituted non-aromatic heterocyclic group.

29. The method of claim 16, wherein R¹, R^{1′}, R², R^{2′}, R³, R^{3′}, R⁴, R^{4′}, R⁶, R⁷, R⁸, R¹⁰, and R¹¹, if present, are in each instance independently hydrogen, halogen, unsubstituted C1-C6 alkyl, hydroxyl, carboxyl, unsubstituted cycloalkyl, unsubstituted C1-C6 alkyloxy, unsubstituted amino, acyl, unsubstituted alkyloxycarbonyl, unsubstituted aryl, unsubstituted heteroaryl, or unsubstituted non-aromatic heterocyclic group.

30. The method of claim 16, wherein R¹, R^{1′}, R², R^{2′}, R³, R^{3′}, R⁴, R^{4′}, R⁶, R⁷, R⁸, R¹⁰, and R¹¹, if present, are in each instance independently hydrogen or halogen.

31. The method of claim 16, wherein R¹, R^{1′}, R², R^{2′}, R³, R^{3′}, R⁴, R^{4′}, R⁶, R⁷, R⁸, R¹⁰, and R¹¹, if present, are each hydrogen.

32. The method of claim 1, wherein the compound has the structure of: 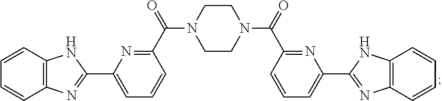 [SMILES: O=C(c1cccc(-c2nc3ccccc3[nH]2)n1)N1CCN(C(=O)c2cccc(-c3nc4ccccc4[nH]3)n2)CC1]
or
a pharmaceutically acceptable salt thereof.

33. The method of claim 1, wherein the compound has the structure of: 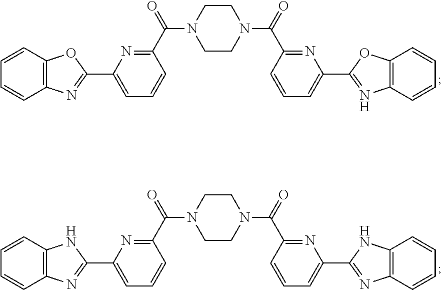 [SMILES: O=C(c1cccc(-c2nc3ccccc3o2)n1)N1CCN(C(=O)c2cccc(-c3nc4ccccc4o3)n2)CC1 ; O=C(c1cccc(-c2nc3ccccc3[nH]2)n1)N1CCN(C(=O)c2cccc(-c3nc4ccccc4[nH]3)n2)CC1]
a pharmaceutically acceptable salt thereof.

34. (canceled)

35. The method of claim 2, wherein the compound has the structure of: 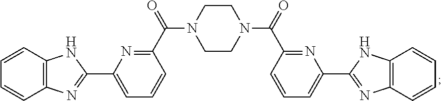 [SMILES: O=C(c1cccc(-c2nc3ccccc3[nH]2)n1)N1CCN(C(=O)c2cccc(-c3nc4ccccc4[nH]3)n2)CC1]
a pharmaceutically acceptable salt thereof.

36. The method of claim 13, wherein the compound has the structure of: 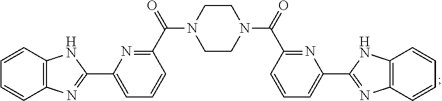 [SMILES: O=C(c1cccc(-c2nc3ccccc3[nH]2)n1)N1CCN(C(=O)c2cccc(-c3nc4ccccc4[nH]3)n2)CC1]
a pharmaceutically acceptable salt thereof.

37. The method of claim 29, wherein the condition comprises at least one of psoriasis, cutaneous lupus erythematosus, dermatomyositis, pemphigoid, pemphigus, scleroderma, vasculitis, epidermolysis bullosa acquisita, vitiligo, lichen planus, scleritis, dermatitis, erythema nodosum, pyoderma gangrenosum, skin fissures, acne, enterocutaneous fistula, skin tags, canker sores, acrodermatitis enteropathica, pyoderma vegetans, leukocytoclastic vasculitis, anal fissures, Sweet's syndrome, rosacea, alopecia, keratoderma blennorrhagica, rosacea, cold sores, urticaria, actinic keratosis, carbuncle, cellulitis, ichthyosis vulgaris, skin infection, malar rash, photosensitivity, livedo reticularis, livedo reticularis, oral and nasal ulcers, purpura, mucositis, hemorrhoids, burn, and sunburn.

38. The method of claim 31, wherein the condition comprises at least one of psoriasis, cutaneous lupus erythematosus, dermatomyositis, pemphigoid, pemphigus, scleroderma, vasculitis, epidermolysis bullosa acquisita, vitiligo, lichen planus, scleritis, dermatitis, erythema nodosum, pyoderma gangrenosum, skin fissures, acne, enterocutaneous fistula, skin tags, canker sores, acrodermatitis enteropathica, pyoderma vegetans, leukocytoclastic vasculitis, anal fissures, Sweet's syndrome, rosacea, alopecia, keratoderma blennorrhagica, rosacea, cold sores, urticaria, actinic keratosis, carbuncle, cellulitis, ichthyosis vulgaris, skin infection, malar rash, photosensitivity, livedo reticularis, livedo reticularis, oral and nasal ulcers, purpura, mucositis, hemorrhoids, burn, and sunburn.

39. The method of claim 29, wherein the condition comprises at least one of psoriasis and atopic dermatitis.

40. The method of claim 31, wherein the condition comprises at least one of psoriasis and atopic dermatitis.
